(19) United States
(12) Patent Application Publication
(10) Pub. No.: US 2016/0040122 A1
(43) Pub. Date: Feb. 11, 2016

(54) NANOFIBERS

(71) Applicant: THE ARIZONA BOARD OF REGENTS ON BEHALF OF THE UNIVERSITY OF ARIZONA, TUCSON, AZ (US)

(72) Inventors: MARVIN SLEPIAN, Tucson, AZ (US); VALERIE MERKLE, Tucson, AZ (US); XIAOYI WU, Tucson, AZ (US)

(21) Appl. No.: 14/818,777

(22) Filed: Aug. 5, 2015

Related U.S. Application Data
(60) Provisional application No. 62/033,450, filed on Aug. 5, 2014.

(51) Int. Cl. C12N 5/00

(52) U.S. Cl. CPC C12N 5/0068; C12N 2537/00; C12N 2533/30; C12N 2533/54

(57) ABSTRACT

The present disclosure relates to nanofibers. In particular, the present disclosure relates to nanofibers comprising multiple layers and their uses in cell culture and tissue engineering.

This application claims the benefit of U.S. provisional application Ser. No. 62/033,450, filed Aug. 5, 2014, which is incorporated herein by reference in its entirety.

### FIELD OF THE INVENTION

The present disclosure relates to nanofibers. In particular, the present disclosure relates to nanofibers comprising multiple layers and their uses in cell culture and tissue engineering.

### BACKGROUND OF THE INVENTION

Traditional electrospinning produces flat, highly interconnected scaffolds consisting of densely packed nanofibers. These electrospun scaffolds can support the adhesion, growth, and function of various cell types, while also promoting their maturation into specific tissue lineages. However, a major limitation of traditional electrospun scaffolds is that they have tightly packed layers of nanofibers with only a superficially porous network, resulting in confinement to sheet-like formations only. This unavoidable characteristic restricts cell infiltration and growth through the scaffolds.

Thus, there is a need to develop new strategies for electrospun scaffolds and their use that overcomes these limitations.

### SUMMARY OF THE INVENTION

The present disclosure relates to nanofibers. In particular, the present disclosure relates to nanofibers comprising multiple layers and their uses in cell culture and tissue engineering. For example, embodiments of the present disclosure provide a composition, comprising: a multi-layer nanofiber comprising a polymeric core and a biocompatible shell. The present disclosure is not limited to a particular polymer. Any degradable or non degradable polymer can be utilized. Examples include, but are not limited to, polymers and copolymers of carboxylic acids such as glycolic acid and lactic acid, polyurethanes, polyesters such as poly(ethylene terephthalate), polyamides such as nylon, polyacrylonitriles, polyphosphazines, polylactones such as polycaprolactone, and polyanhydrides such as poly[bis(p-carboxphenoxy)propane anhydride] and other polymers or copolymers such as polyethylene, polyvinyl chloride and ethylene vinyl acetate, homopolymers and copolymers of delta-valerolactone, and p-dioxanone as well as their copolymers with caprolactone, and those described in U.S. Pat. No. 6,290,729, herein incorporated by reference in its entirety. In some exemplary embodiments, the polymer is PVA.

The present disclosure is not limited to particular biocompatible material. Examples include, but are not limited to, large molecular weight proteins (e.g., gelatin, collagen, fibrin, fibrinogen, albumin, laminin, zein, etc.), lipids, phospholipids, and glycoproteins.

In some embodiments, the biocompatible layer comprises or is coated with an agent that alters the surface texture or provides a biological function (See e.g., Tran et al., Advanced Healthcare Materials Volume 2, Issue 7, page 1064, July, 2013; herein incorporated by reference in its entirety). Examples include but are not limited to, ligands (e.g. peptide ligands), nanoparticles, iron, labels, contrast agents, cells, encapsulated particles, and viruses.

In some embodiments, agents provide a biological function (e.g., differentiation of cells such as stem cells, etc.). In some embodiments, agents provide a label or contrast agent for analysis or imaging of cells or tissues growing on the nanofibers.

In some embodiments, agents are mixed with the outer biocompatible layer of the nanofiber and applied using electrospinning (See e.g., Bae et al., Journal of Nanoscience and Nanotechnology, Volume 14, Number 10, October 2014, pp. 7574-7580(7); herein incorporated by reference in its entirety). In some embodiments, agents are added after particle formation.

The multilayered nanofibers of the present disclosure comprise two or more (e.g., three or more, four or more, five or more, etc.) layers of polymeric and/or biocompatible materials. In some embodiments, the particles comprise one or more polymeric layers covered by one or more biocompatible material layers.

In some exemplary embodiments, PVA/gelatin core-shell nanofibers are utilized. In some embodiments, the PVA and the gelatin are present at a ratio of approximately 1:1 or 1:3 in the nanofiber, although other ratios are contemplated. In some embodiments, the nanofiber is made by electrospinning. In some embodiments, the nanofibers exhibit an increased Young's modulus, a higher tensile strength, or reduced plastic deformation relative to PVA nanofibers.

In further embodiments, the present disclosure provides a system, comprising: a) any of the aforementioned nanofiber; and b) a plurality of cells, tissues or an organ in operable communication with a surface of the nanofiber. The present disclosure is not limited to particular cells, tissues, or organs. The nanofibers described herein are suitable for use in the culture or growth of any number of prokaryotic and eukaryotic cells. Examples of cells include, but are not limited to, islet cells, fibroblasts, hormone secreting cells, stem cells (e.g. adipose-derived stem cells) neurons, epithelial cells, blood cells, or blood cell products (e.g., platlets). Examples of tissues include, but are not limited to, connective tissue, muscle tissue, nervous tissue, or epithelial tissue. Examples of organs include, but are not limited to, heart, stomach, liver, gallbladder, pancreas, intestines, colon, rectum, anus, hypothalamus, pituitary gland, pineal body or pineal gland, thyroid, parathyroids, adrenals, kidneys, tonsils, adenoids, thymus, spleen, skin, hair, nails, brain, spinal cord, nerves, ovaries, fallopian tubes, uterus, vagina, mammary glands, testes, vas deferens, seminal vesicles, prostate, penis, pharynx, larynx, trachea, bronchi, lungs, diaphragm, bones, cartilage, ligaments or tendons.

Additional embodiments provide a method of culturing cells, tissues, or organs, comprising: a) contacting a cell, tissue, or organ with a surface of any of the aforementioned core-shell nanofibers; and b) culturing the cell, tissue, or organ.

Additional embodiments are described herein.

### DESCRIPTION OF THE DRAWINGS

FIG. 1 shows nanofiber fabrication. SEM images of (a) gelatin, (c) PVA, (e) 1:1 PVA/gelatin coreshell, and (g) 1:3 PVA/gelatin core-shell nanofibers. Fiber diameter distributions of (b) gelatin, (d) PVA, (f) 1:1 PVA/gelatin core-shell, and (h) 1:3 PVA/gelatin core-shell nanofibers.

FIG. 2 shows TEM images of (a) 1:1 PVA/gelatin core-shell nanofibers and (b) 1:3 PVA/gelatin coreshell nanofibers.

FIG. 3 shows FTIR spectra of (a) crosslinked gelatin, (b) uncrosslinked gelatin, (c) crosslinked 1:1 PVA/gelatin core-shell, (d) crosslinked 1:3 PVA/gelatin core-shell, (e) crosslinked PVA, and (f) uncrosslinked PVA nanofibers.

FIG. 4 shows representative stress-strain curves of gelatin, PVA, 1:1 PVA/gelatin core-shell, and 1:3 PVA/gelatin core-shell randomly oriented nanofibers. All the specimens were crosslinked.

FIG. 5 shows (a) SEM images of PVA, 1:1 PVA/gelatin core-shell and 1:3 PVA/gelatin core-shell aligned nanofibers. (b) Representative stress-strain curves of PVA, 1:1 PVA/gelatin core-shell and 1:3 PVA/gelatin core-shell aligned nanofibers. All the specimens were crosslinked.

FIG. 6 shows adhesion profiles of NIH/3T3 fibroblasts on the PVA, 1:1 PVA/gelatin core-shell, 1:3 PVA/gelatin core-shell, and gelatin scaffolds.

FIG. 7 shows proliferation profiles of NIH/3T3 fibroblasts grown on the gelatin films, gelatin scaffolds, PVA scaffolds, 1:1 PVA/gelatin coreshell scaffolds, 1:3 PVA/gelatin core-shell scaffolds, and the TCP up to 7 days. The optical density of the cells grown on each type of scaffolds was normalized by that of the cells grown on the TCP on day 1.

FIG. 8 shows fluorescent staining for cell viability of NIH/3T3 fibroblasts grown on (a) gelatin, (b) PVA, (c) 1:1 PVA/gelatin, and (d) 1:3 PVA/gelatin core-shell nanofiber scaffolds for 3 days.

FIG. 9 shows electrospinning of coreshell nanofibers. SEM (a) and TEM images (b) of the coaxially electrospun PVA/gelatin coreeshell nanofibers.

FIG. 10 shows representative tensile stress-strain curves of the gelatin nanofiber mats (a), the PVA nanofiber mats (b), the gelatin/PVA coreshell nanofiber mats (c), and the PVA/gelatin coreshell nanofiber mats (d).

FIG. 11 shows representative SEM images (a) and tensile stress-strain curves (b) of the aligned PVA nanofiber mats and the gelatin/PVA coreshell aligned nanofiber mats.

FIG. 12 shows FT-IR spectra of the gelatin nanofibers (a), the PVA/gelatin coreshell nanofibers (b), and the PVA nanofibers (c).

FIG. 13 shows an exemplary mechanism for the observed mechanical strengthening effects. (a) In the PVA/gelatin coreshell nanofibers, the molecular alignment of semi-crystalline PVA is enhanced by the protecting gelatin shell. (b) In the reverse coreshell nanofibers, no enhancement in the alignment of molecules in the PVA shell is expected.

FIG. 14 shows adipose-derived stem cell morphology on electrospun scaffolds of (A) gelatin (B) PVA (C) 1 Gel:1 PVA coaxial (D) 3 Gel:1 PVA coaxial.

FIG. 15 shows adipose-derived stem cell morphology on electrospun scaffolds of gelatin, PVA, 1 Gel:1 PVA coaxial, and 3 Gel:1 PVA coaxial.

FIG. 16 shows adipose-derived stem cell viability on electrospun scaffolds-gelatin, PVA, 1 Gel:1 PVA coaxial, 3 Gel:1 PVA coaxial, gelatin film, and tissue culture plate after 1, 3, 5, and 7 days after initial cell seeding.

FIG. 17 shows adipose-derived stem cell adhesion to electrospun scaffolds of gelatin, PVA, 1 Gel:1 PVA coaxial, and 3 Gel:1 PVA coaxial, as well as the tissue culture plate.

FIG. 18 shows Adipose-derived stem cell proliferation over 7 days of incubation on electrospun gelatin, PVA, 1 Gel:1 PVA coaxial, and 3 Gel:1 PVA coaxial scaffolds.

FIG. 19 shows adipose-derived stem cell proliferation on the scaffold surface at day 1, 3, 5, and 7 after initial cell seeding on the gelatin scaffolds, PVA scaffolds, 1 Gel:1 PVA scaffolds, 3 Gel:1 PVA scaffolds, and TCP.

FIG. 20 shows migration of adipose-derived stem cells on electrospun gelatin, PVA, 1 Gel:1 PVA coaxial, 3 Gel:1 PVA coaxial, and tissue culture plate (A) without growth factor (B) with growth factor

FIG. 21 shows adipose-derived stem cell outward migration (no growth factor present) on gelatin, PVA, 1 Gel:1 PVA coaxial, 3 Gel:1 PVA coaxial scaffolds, as well as the tissue culture plate. (A) Area on the scaffolds occupied by cells over 48 hours (B) Percent outward migration at each time point.

FIG. 22 shows adipose-derived stem cell outward migration (with growth factor present) on gelatin, PVA, 1 Gel:1 PVA coaxial, 3 Gel:1 PVA coaxial scaffolds, as well as the gelatin film and tissue culture plate. (A) Area on the scaffolds occupied by cells over 48 hours (B) Percent outward migration at each time point

FIG. 23 shows outward migration rate of adipose-derived stem cells on electrospun scaffolds-gelatin, PVA, 1 Gel:1 PVA coaxial, 3 Gel:1 PVA coaxial, and tissue culture plate-over 48 hours with and without growth factor present.

FIG. 24 shows fiber surface roughness assessed using atomic force microscopy (AFM) for (A) gelatin scaffolds, (B) PVA scaffolds, (C) 1 Gel:1 PVA coaxial scaffolds, and (D) 3 Gel:1 PVA coaxial scaffolds. Surface roughness values are presented as Ra (nm) in the table (panel E).

FIG. 25 shows platelet viability assessed using a lactate dehydrogenase (LDH) assay after 3 h of seeding on gelatin scaffolds, PVA scaffolds, 1 Gel:1 PVA coaxial scaffolds, 3 Gel:1 PVA coaxial scaffolds, and tissue culture plate (TCP).

FIG. 26 shows platelet deposition under static conditions on electrospun scaffolds (A) gelatin, (B) PVA, (C) 1 Gel:1 PVA coaxial, and (D) 3 Gel:1 PVA coaxial. (E). Scale bar is 20 μm.

FIG. 27 shows (A) Platelet activation state (PAS) and (B) platelet activation rate (PAR) of platelets incubated under static conditions on the scaffolds (gelatin, PVA, 1 Gel:1 PVA coaxial, and 3 Gel:1 PVA coaxial) and the tissue culture plate (TCP).

FIG. 28 shows deposition of platelets preactivated (A-D) mechanically (HSD) or (E-H) chemically (ADP) on defined surfaces: gelatin scaffolds (A, E), PVA scaffolds (B, F), 1 Gel:1 PVA coaxial scaffolds (C, G), and 3 Gel:1 PVA coaxial scaffolds (D, H).

FIG. 29 shows platelet deposition on surfaces preseeded with smooth muscle cells (SMC) (A-D) or human umbilical vein endothelial cells (HUVEC) (E-H) on gelatin scaffolds (A, E), PVA scaffolds (B, F), 1 Gel:1 PVA coaxial scaffolds (C, G), and 3 Gel:1 PVA coaxial scaffolds (D, H).

FIG. 30 shows platelet deposition on electrospun scaffolds under shear, 1 dyn/cm2 (A-D) and 3 dyn/cm2 (E-H). Platelet deposition was calculated on the following scaffolds per high powered field (3000×): gelatin scaffolds (A, E), PVA scaffolds (B,F), 1 Gel:1 PVA coaxial scaffolds (C, G), 3 Gel:1 PVA coaxial scaffolds (D, H). Platelet deposition on gelatin, PVA, 1 Gel:1 PVA coaxial, or 3 Gel:1 PVA coaxial scaffolds under shear (1 or 3 dyn/cm2) is presented in this bar graph per high-powered field (3000×) (I).

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “solvent” refers to a medium in which a reaction is conducted. Solvents may be liquid but are not limited to liquid form. Solvent categories include but are not limited to nonpolar, polar, protic, and aprotic.

As used herein, “electrospun material” is any molecule or substance that forms a structure or group of structures (e.g., fibers, webs, or droplet), as a result of the electrospinning process. This material may be natural, synthetic, or a combination of such.

“Polymer” is any natural or synthetic molecule which can form long molecular chains, such as polyolefin, polyamides, polyesters, polyurethanes, polypeptides, polysaccharides, and combinations thereof. In particular, the polymer can include: poly(epsilon.-caprolactone), poly vinyl alcohol, polylactic acid, poly(lactic-co-glycolic) acid, poly(etherurethane urea), collagen, elastin, chitosan, or any combination of these.

### DETAILED DESCRIPTION OF THE INVENTION

The present disclosure relates to nanofibers. In particular, the present disclosure relates to nanofibers comprising multiple layers and their uses in cell culture and tissue engineering.

Electrospinning as a facile and universal fiber-forming technique has enabled the fabrication of a variety of biomaterials into micro/nanometer-diameter fibers, including synthetic polymers (Reneker, D. H. et al., Nanotechnology 1996, 7, 216-223; Li, D. et al., Adv Mater 2004, 16, 1151-1170), proteins (Li, C. M. et al., Biomaterials 2006, 27, 3115-3124; Li, M. Y. et al., Biomaterials 2005, 26, 5999-6008) and lipids (McKee, M. G. et al, Science 2006, 311, 353-355; Zha, Z. B. et al., Adv Mater 2011, 23, 3435-3440). The high specific surface areas, porosities, and micro/nanoscale features of electrospun fibers provide new and/or enhanced functions that are not obtainable from bulk materials and are thus useful for applications in tissue engineering, drug delivery, wound healing, and medical implants. Efforts have further endeavored to electrospin dual- or multiple-component fibers with a new set of properties and/or functions that are unattainable from single components. Such composite nanofibers overcome the inherent limitations of single-component fibers. For instance, an implantable scaffold that enables the in vivo regeneration of vascular tissues needs both mechanical strength and biological functions (Naito, Y. et al., Adv Drug Delivery Rev 2011, 63, 312-323; Tillman, B. et al., Biomaterials 2009, 30, 583-588).

Scaffolds composed of synthetic polymers often have desirable mechanical properties but display minimal bioactivity because of their insufficient cellular recognition sites (Feng, W. et al., Adv Biosci Biotechnol 2010, 2010, 185-189; Linh, N. T. et al., J Biomater Appl 2012, 27, 255-266; Linh, N. T. B. et al., J Biomed Mater Res Part B 2010, 95B, 184-191; Nien, Y. H. et al., J Med Biol Eng 2009, 29, 98-101). In contrast, scaffolds composed solely of natural proteins tend to have high bioactivity but lack the mechanical strength needed for in vivo applications (Feng, W. et al., Adv Biosci Biotechnol 2010, 2010, 185-189; Linh, N. T. et al., J Biomater Appl 2012, 27, 255-266; Linh, N. T. B. et al., J Biomed Mater Res Part B 2010, 95B, 184-191; Nien, Y. H. et al., J Med Biol Eng 2009, 29, 98-101; Sisson, K. et al., Biomacromolecules 2009, 10, 1675-1680; Zha, Z. et al., Biopolymers 2012, 97, 1026-1036). The fabrication of composite nanofiber scaffolds of embodiments of the present disclosure that integrate the mechanical strength of synthetic polymers and the bioactivity of natural proteins address the unmet need in vascular tissue engineering.

Accordingly, embodiments of the present disclosure provide multilayer (e.g., core-shell) nanofibers comprising a polymer (e.g., polyvinyl alcohol (PVA)) core and a biocompatible (e.g., gelatin) shell or outer layer.

The present disclosure is not limited to a particular polymer. Any degradable or non degradable polymer can be utilized. Examples include, but are not limited to, polymers and copolymers of carboxylic acids such as glycolic acid and lactic acid, polyurethanes, polyesters such as poly(ethylene terephthalate), polyamides such as nylon, polyacrylonitriles, polyphosphazines, polylactones such as polycaprolactone, and polyanhydrides such as poly[bis(p-carboxphenoxy)propane anhydride] and other polymers or copolymers such as polyethylene, polyvinyl chloride and ethylene vinyl acetate, homopolymers and copolymers of delta-valerolactone, and p-dioxanone as well as their copolymers with caprolactone, and those described in U.S. Pat. No. 6,290,729, herein incorporated by reference in its entirety. In some exemplary embodiments, the polymer is PVA.

The present disclosure is not limited to particular biocompatible material. Examples include, but are not limited to, large molecular weight proteins (e.g., gelatin, collagen, fibrin, fibrinogen, albumin, laminin, zein, etc.), lipids, phospholipids, and glycoproteins.

In some embodiments, the biocompatible layer comprises or is coated with an agent that alters the surface texture or provides a biological function (See e.g., Tran et al., Advanced Healthcare Materials Volume 2, Issue 7, page 1064, July, 2013; herein incorporated by reference in its entirety). Examples include but are not limited to, ligands (e.g. peptide ligands), nanoparticles, iron, labels, contrast agents, cells, encapsulated particles, and viruses.

In some embodiments, agents provide a biological function (e.g., differentiation of cells such as stem cells, etc.). In some embodiments, agents provide a label or contrast agent for analysis or imaging of cells or tissues growing on the nanofibers.

In some embodiments, agents are mixed with the outer biocompatible layer of the nanofiber and applied using electrospinning (See e.g., Bae et al., Journal of Nanoscience and Nanotechnology, Volume 14, Number 10, October 2014, pp. 7574-7580(7); herein incorporated by reference in its entirety). In some embodiments, agents are added after particle formation.

The multilayered nanofibers of the present disclosure comprise two or more (e.g., three or more, four or more, five or more, etc.) layers of polymeric and/or biocompatible materials. In some embodiments, the particles comprise one or more polymeric layers covered by one or more biocompatible material layers.

In some exemplary embodiments, PVA/gelatin core-shell nanofibers are utilized. In some embodiments, the ratio of PVA to gelatin in the nanofiber is approximately 1:1, 1:3 or other ratios in-between 1:1 and 1:3, although other ratios are specifically contemplated. The PVA/gelatin nanofibers of embodiments of the present disclosure exhibit improved Young's modulus and tensile strength relative to PVA nanofibers and are suitable for cell, tissue, or organ culture. Thus, the nanofibers of embodiments of the present disclosure provide improved strength in a fiber for use as a scaffold for biological applications.

The nanofibers are made by any suitable method. In some embodiments, electrospinning is utilized to generate core-shell nanofibers of embodiments of the present disclosure (See e.g., U.S. Pat. No. 8,257,641; herein incorporated by reference in its entirety). The process of electrospinning involves placing a polymer-containing fluid (for example, a polymer solution, a polymer suspension, or a polymer melt) in a reservoir equipped with a small orifice, such as a needle or pipette tip and a metering pump. One electrode of a high voltage source is also placed in electrical contact with the polymer-containing fluid or orifice, while the other electrode is placed in electrical contact with a target (typically a collector screen or rotating mandrel). During electrospinning, the polymer-containing fluid is charged by the application of high voltage to the solution or orifice (for example, about 3-15 kV) and then forced through the small orifice by the metering pump that provides steady flow. While the polymer-containing fluid at the orifice normally would have a hemispherical shape due to surface tension, the application of the high voltage causes the otherwise hemispherically shaped polymer-containing fluid at the orifice to elongate to form a conical shape known as a Taylor cone. With sufficiently high voltage applied to the polymer-containing fluid and/or orifice, the repulsive electrostatic force of the charged polymer-containing fluid overcomes the surface tension and a charged jet of fluid is ejected from the tip of the Taylor cone and accelerated towards the target, which typically is biased between −2 to −10 kV. Optionally, a focusing ring with an applied bias (for example, 1-10 kV) can be used to direct the trajectory of the charged jet of polymer-containing fluid. As the charged jet of fluid travels towards the biased target, it undergoes a complicated whipping and bending motion. If the fluid is a polymer solution or suspension, the solvent typically evaporates during mid-flight, leaving behind a polymer fiber on the biased target. If the fluid is a polymer melt, the molten polymer cools and solidifies in mid-flight and is collected as a polymer fiber on the biased target. As the polymer fibers accumulate on the biased target, a non-woven, porous mesh is formed on the biased target.

The properties of the nanofibers can be tailored by varying the electrospinning conditions. For example, when the biased target is relatively close to the orifice, the resulting electrospun mesh tends to contain unevenly thick fibers, such that some areas of the fiber have a “bead-like” appearance. However, as the biased target is moved further away from the orifice, the fibers of the non-woven mesh tend to be more uniform in thickness. Moreover, the biased target can be moved relative to the orifice. In certain non-limiting embodiments, the biased target is moved back and forth in a regular, periodic fashion, such that fibers of the non-woven mesh are substantially parallel to each other. When this is the case, the resulting non-woven mesh may have a higher resistance to strain in the direction parallel to the fibers, compared to the direction perpendicular to the fibers. In other non-limiting embodiments, the biased target is moved randomly relative to the orifice, so that the resistance to strain in the plane of the non-woven mesh is isotropic. The target can also be a rotating mandrel. In this case, the properties of the non-woven mesh may be changed by varying the speed of rotation. The properties of the electrospun elastomeric scaffold may also be varied by changing the magnitude of the voltages applied to the electrospinning system. In one non-limiting embodiment, the electrospinning apparatus includes an orifice biased to 12 kV, a target biased to −7 kV, and a focusing ring biased to 3 kV. Moreover, a useful orifice diameter is 0.047″ (I.D.) and a useful target distance is about 23 cm. Other electrospinning conditions that can be varied include, for example and without limitation, the feed rate of the polymer solutions, the solution concentrations, and the polymer molecular weight.

The nanofibers described herein find use in a variety of research, screening, and therapeutic applications. In some embodiments, the nanofibers find use in cell culture, cell delivery, tissue culture, organ culture, drug screening and/or drug delivery applications.

The nanofibers find use in the culture of a variety of cells, tissues, and organs. Examples of cells include, but are not limited to, islet cells, fibroblasts, hormone secreting cells, neurons, epithelial cells, blood cell products (e.g., platelets), stem cells, or blood cells. Examples of tissues include, but are not limited to, connective tissue, muscle tissue, nervous tissue, or epithelial tissue. Examples of organs include, but are not limited to, heart, stomach, liver, gallbladder, pancreas, intestines, colon, rectum, anus, hypothalamus, pituitary gland, pineal body or pineal gland, thyroid, parathyroids, adrenals, kidneys, tonsils, adenoids, thymus, spleen, skin, hair, nails, brain, spinal cord, nerves, ovaries, fallopian tubes, uterus, vagina, mammary glands, testes, vas deferens, seminal vesicles, prostate, penis, pharynx, larynx, trachea, bronchi, lungs, diaphragm, bones, cartilage, ligaments or tendons.

### EXPERIMENTAL

The following examples are provided in order to demonstrate and further illustrate certain preferred embodiments and aspects of the present invention and are not to be construed as limiting the scope thereof.

### Example 1

### Materials

PVA (MW: 89-98 kDa, 99+% hydrolyzed), gelatin type A, 25% aqueous glutaraldehyde (GTA), and dimethyl sulfoxide were obtained from Sigma Aldrich. Phosphate-buffered saline (10×PBS) was a product of Hyclone. Ethanol (200 proof) was purchased from Decon Laboratories. Deionized water was also used.

Scaffold Fabrication

A custom-built electrospinning apparatus (Qiu, W. G. et al., Biomacromolecules 2010, 11, 3219-3227) was used to complete all electrospinning A 16% w/v solution of PVA was prepared with ethanol and deionized water (1:9 v/v) in a water bath for 4 h at 60° C. To electrospin PVA, the polymeric solution was placed in a 1 mL plastic syringe with a needle tip diameter of 0.25 mm (inner diameter). PVA fibers were fabricated using an applied voltage of 12 kV (Acopian High Voltage Power Supply) and a fixed needle tip to grounded collector distance of 12 cm. A syringe pump (Kazel Syringe Pumps, R-99) fed the polymer solution through the needle tip at a fixed rate of 9 mL/min. Gelatin Type A was dissolved with ethanol and 10×PBS (1:1 v/v) in a water bath for 2 h at 40° C. The 15% w/v gelatin solution was then kept at room temperature for 12 h for further dissolution. The polymeric solution was then placed in a 1 mL plastic syringe with a needle tip diameter of 0.33 mm (inner diameter). Gelatin fibers were fabricated using an applied voltage of 12 kV with a fixed needle tip to grounded collector distance of 12 cm. A syringe pump fed the polymer solution through the needle tip at a fixed rate of 30 μL/min.

A custom-built coaxial set-up was constructed to electrospin fibers that contained both PVA and gelatin. The previously described solutions of gelatin and PVA were prepared and placed in their respective 1 mL syringes. For coaxial electrospinning, the needle tip of the inner needle was 0.25 mm (inner diameter), whereas the outer needle tip was 0.84 mm (inner diameter). A high voltage of 15 kVor 20 kV was applied for PVA and gelatin at a 1:1 and 1:3 volume ratio, respectively. The ground collector was located at a fixed needle tip to collector distance of 12 cm (for 1:1 PVA/gelatin) and 15 cm (for 1:3 PVA/gelatin). A syringe pump fed the respective polymeric solutions through the coaxial set-up at a fixed flow rate (7 μL/min for 1:1 PVA/gelatin and 3 μL/min and 9 μL/min for 1:3 PVA/gelatin).

Aligned nanofibers were electrospun using a gap method (Li, D. et al., Core-Shell Nanofibers 345 Biopolymers). In this method, a stainless steel plate with a rectangular-shaped hole cut in the center was used as the grounded collector. Electrospinning parameters and solutions were used as previously described. Aligned nanofibers were deposited bridging the gap on the stainless steel plate.

The PVA, gelatin, and coaxial nanofibers, both randomly oriented and aligned nanofibers, were all crosslinked using a 0.5% GTA aqueous solution (Zha, Z. et al., Biopolymers 2012, 97, 1026-1036; Qiu, W. G. et al., Biomacromolecules 2010, 11, 3219-3227). The scaffolds were vacuum sealed in a desiccator for 20 h at a fixed temperature of 42° C.

### Scaffold Characterization

The surface morphology of the nanofibrous scaffolds was studied using a Hitachi-S4800 field emission SEM (Qiu, W. G. et al., Biomacromolecules 2010, 11, 3219-3227). Before microscopic observation, the scaffolds were air-dried for 24 h, mounted, and coated with platinum for 30 s in a sputtercoater. Image J (supported by the National Institutes of Health) was used to calculate the fiber diameter and pore size of randomly oriented fibers from 10 different images of each sample. The fiber diameter distribution for each sample was then calculated from more than 300 randomly selected fibers. A FEI CM128 TEM, operated at 80 kV, was used to obtain images of the core-shell structure of the nanofibers. The nanofibers were collected on a copper grid prior to imaging. Eight bit TIFF digital images were collected by an AMT 4 M pixel camera.

FTIR spectra were obtained for the crosslinked gelatin scaffolds, uncrosslinked gelatin scaffolds, crosslinked PVA scaffolds, uncrosslinked PVA scaffolds, 1:1 PVA/gelatin composite scaffolds, and 1:3 PVA/gelatin composite scaffolds. The samples were prepared on zinc-selenium windows. A Nicolett Magna IR 560 Spectrometer equipped with a MCT/A Detector (liquid nitrogen cooled) recorded the FTIR spectra of these samples. FTIR spectra were obtained within the range of 4000 to 650 cm⁻¹ using 128 scans with a corresponding resolution of 2 cm⁻¹.

A PerkinElmer dynamic mechanical analyzer (DMA) was used for a uniaxial tensile analysis of the crosslinked scaffolds. The scaffolds were cut into rectangular strips whose thickness was determined using an optical microscope. The samples were mounted in the DMA with a gauge length of 5 mm in an ambient environment. The samples were stretched monotonically until failure at a constant rate of 0.1 mm/min. From the stress-strain curve, the Young's modulus, ultimate tensile strength, and strain at failure of each type of scaffold material were calculated.

### Cell Culture

To prepare the scaffolds for cell culture, the scaffolds were placed in an oven at 42° C. for a period of 24 h to remove residual GTA from the post fabrication treatment. Next, the scaffolds were exposed to ultraviolet light for 45 min to sterilize them for cell culture. After sterilization, the scaffolds were allowed to soak in Dulbecco's modified eagle medium supplemented with 10% v/v fetal bovine serum and 0.05 g/mL gentamicin for a period of 30 min before cell seeding. The scaffolds, immersed in the media, were then seeded with NIH 3T3 fibroblasts and incubated in an incubator at 37° C., 5% CO₂, and 95% relative humidity. As a control, cells were seeded on the tissue culture polystyrene well without any scaffolds present.

The scaffolds were prepared for SEM analysis 2 days after seeding NIH 3T3 fibroblasts onto a scaffold in a 24-well plate. Briefly, the samples were submerged in a primary fixation solution of 4% paraformaldehyde, rinsed with 1×PBS, dehydrated through a series of increasing ethanol concentrations, and finally, dried with a Polaron critical point drier using liquid carbon dioxide. Each sample was coated with gold and imaged using a Hitachi-S4800 SEM.

### Cell Adhesion.

NIH 3T3 fibroblasts were seeded onto the scaffold surface and incubated for 3.5 h to allow the fibroblasts to attach to the scaffold. After incubation, the medium was aspirated from each well and the scaffolds rinsed three times with PBS to remove any cells that were not attached to the scaffold. After rinsing, cell media and MTT (thiazolyl blue tetrazolium bromide) solution (5 mg/mL in 13 PBS) were added to each well and then incubated for 3.5 h at 37° C. and 5% CO₂. The mitochondria of living cells reacted with the tetrazolium salt in the MTT reagent to yield a soluble formazan dye. The absorbance of each well was quantified using a Nanodrop UV-vis spectrophotometer (Thermo Scientific) at a wavelength of 630 nm. The tissue culture polystyrene plate with the culture medium was used as the negative control for this study. Cell adhesion was defined by the following relationship:

% cell adhesion=100×absorbance of sample/absorbance of control

### Cell Proliferation.

Cell proliferation of NIH 3T3 fibroblasts was determined using a MTT assay with the protocol described in the preceding section. Cell culture plates were analyzed at day 1, day 3, day 5, and day 7 after initial cell seeding, with the cell culture media being refreshed every 2 days.

### Cell Viability.

Cellular viability on the electrospun scaffolds was determined using a Live/Dead Cytotoxicity Kit (Invitrogen) 3 days after initial cell seeding. Each scaffold/well was initially seeded with 3T3 fibroblasts and incubated for a period of 3 days. Briefly, the culture medium was aspirated from each of the wells, the samples rinsed three times with 13 PBS, and a working solution (consisting of 5 mL 13 PBS, 10 μL 2 mM EthD-1, and 2.5 μL-4 mM Calcein AM) added to cover each of the samples. The scaffolds submerged in the working solution were incubated for 30 min. After the incubation period, the working solution was removed, and the samples were rinsed once with 1×PBS. Lastly, images were taken using a fluorescent microscope (Leica DMI 4000B).

### Statistical Analysis

All mechanical data were presented as mean±standard deviation. All cellular data were expressed as mean±standard error. Statistical significance of differences between the means was determined using Student's t-test.

### Results

### Formation of Core-Shell Nanofibers

Previous studies have shown coaxial electrospinning of polycaprolactone (PCL)/gelatin core-shell fiber composites with the aim of increasing the bioactivity of PCL (Zhang, Y. Z. et al., Biomacromolecules 2005, 6, 2583-2589; Zhao, P. C. et al., J Biomed Mater Res Part A 2007, 83A, 372-382). These studies show that fibroblasts have better proliferation on the PCL/gelatin core-shell fibers than fibers composed of solely PCL. Use of an organic solvent is useful for dissolution of PCL; therefore, for coaxial PCL/gelatin fiber fabrication, each material was dissolved in trifluoroethanol to ensure formation of the core-shell structure. Often, organic solvents such as triflouroethanol (Chong, E. J. et al., Acta Biomater 2007, 3, 321-330) or hexaflouropropanol (Kim, H. W. et al., Adv Funct Mater 2005, 15, 1988-1994) are used to electrospin gelatin nanofibers. However, residual organic solvents remaining on the fibers are toxic to seeded cells. Recently, the use of mixed PBS and ethanol as a benign solvent for the electrospinning of gelatin nanofibers was proposed (Zha, Z. et al., Biopolymers 2012, 97, 1026-1036). To fabricate the core-shell nanofibers, PVA was dissolved in a mixture of ethanol and de-ionized water at a 1:9 volume ratio, and ethanol and PBS in a 1:1 volume ratio was used for the dissolution of gelatin. Although PVA nanofibers can be electrospun from an aqueous solution, 16 the inclusion of ethanol increases the evaporability of the PVA solution throughout the electrospinning process, minimizing the occurrence of fiber deficiencies. The use of ethanol/deionized water as the PVA solvent and ethanol/PBS as the gelatin solvent provides a benign solvent system to promote cell viability on yielded nanofibers. In addition, the similar evaporability of the two solvents facilitates the formation of core-shell nanofibers. Consequently, PVA/gelatin core-shell composite nanofibers were electrospun at a 1:1 and 1:3 PVA/gelatin mass ratio, respectively.

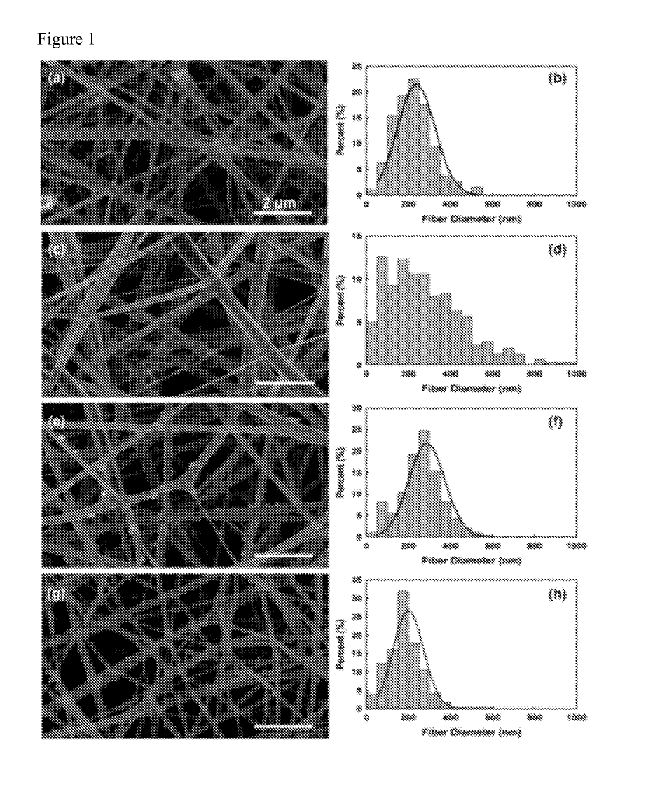

SEM images showed uniform fiber morphology for the gelatin, PVA, 1:1 PVA/gelatin, and 1:3 PVA/gelatin composite scaffolds, respectively (FIG. 1). A quantitative analysis of the SEM images revealed fiber diameters of the scaffolds as follows (Table I): 223±93 nm (gelatin), 283±188 nm (PVA), 256±99 nm (1:1 PVA/gelatin), and 182±81 nm (1:3 PVA/gelatin). Further, the gelatin and core-shell nanofiber scaffolds displayed a normal Gaussian distribution of fiber diameter, whereas the PVA fiber diameter distribution showed two peaks. This was due to fiber branching and bifurcation that yielded secondary, small-diameter fibers (<100 nm) in addition to primary, large-diameter nanofibers (>250 nm). Because gelatin was dissolved in PBS, nanometer-sized salt crystals appeared on the gelatin and PVA/gelatin core-shell scaffolds and were readily removed by washing the crosslinked scaffolds in water.

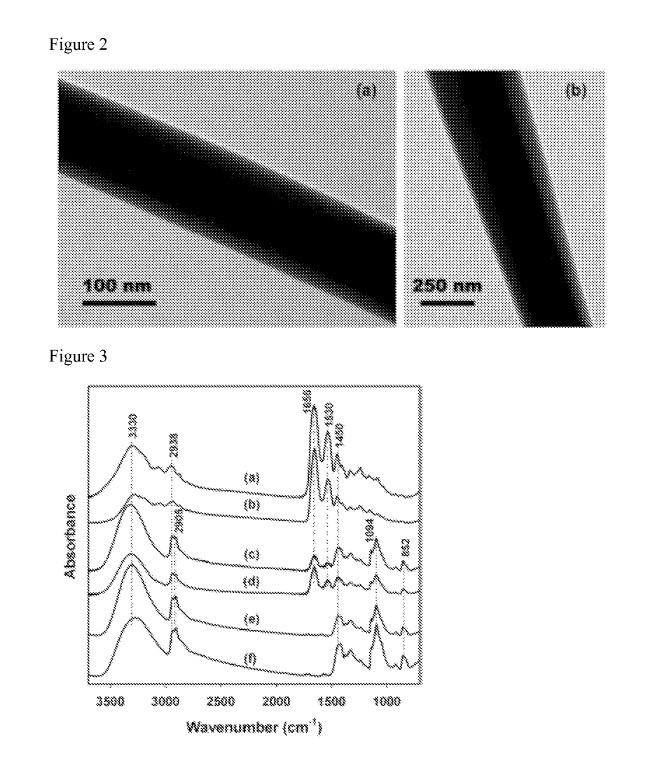

TEM was used to visualize the core-shell structure of the composite nanofibers. FIG. 2 illustrates that gelatin is in the shell position and PVA is in the core position of both the 1:1 PVA/gelatin and 1:3 PVA/gelatin composite nanofibers. Further, the 1:1 PVA/gelatin composite nanofibers show a thin gelatin sheath, whereas the 1:3 PVA/gelatin composite nanofibers possess a thicker gelatin sheath, due to the higher gelatin/PVA mass ratio.

### FTIR Spectroscopy of Electrospun Scaffolds

Potential changes in the primary and secondary structures of the electrospun scaffolds through electrospinning were analyzed using FTIR spectroscopy. The fabricated scaffolds were crosslinked using 0.5% GTA vapor in a vacuum oven at 42° C. for 20 h. Studies have indicated that the use of 0.5% GTA vapor decreases the inherent cytotoxicity present with GTA.13 FTIR spectra were obtained for crosslinked and uncrosslinked gelatin scaffolds, crosslinked and uncrosslinked PVA scaffolds, crosslinked 1:1 PVA/gelatin composite scaffolds, and crosslinked 1:3 PVA/gelatin composite scaffolds (FIG. 3). The spectra of the crosslinked and uncrosslinked gelatin scaffolds possessed peaks at 3285-3305 cm⁻¹ (amide A), 3060 cm⁻¹ (amide B), 2938 cm⁻¹ (CH2 stretching), 1655 cm⁻¹ (amide I), 1530 cm⁻¹ (amide II), 1450 cm⁻¹ (CH₂ scissoring and CH₃ asymmetric bending), 1406 cm⁻¹ (CH₂ wagging), 1240 cm⁻¹ (amide III), and 1158 cm⁻¹ (CC stretching) (Lai, G. L. et al., Korea-Aust Rheol J 2007, 19, 81-88; Cao, H. et al., Food Chem 2008, 108, 439-445). Previously, it was reported that the electrospinning process partially converted gelatin from a less-ordered, β-turn conformation to a more-ordered, α-helix structure (Zha et al., Biopolymers 2012, 97, 1026-1036). This study shows that the GTA crosslinking process slightly shifted the amide I band from 1654 cm⁻¹ to 1657 cm⁻¹, typical of an a-helix structure, and largely preserved the a-helix structure. The spectra of crosslinked and uncrosslinked PVA scaffolds displayed peaks at 3000-3600 cm⁻¹ (OH stretching), 2935/2905 cm⁻¹ (CH₂ asymmetric and symmetric stretching), 1426 cm⁻¹ (CH₂ bending), 1094 cm21 (CAO stretching), and 852 cm⁻¹ (CH bending). 9,30,31

The spectra of the crosslinked PVA/gelatin core-shell scaffolds showed peaks characteristic of PVA and gelatin, indicating the presence of both materials in the composite nanofibers. Further, no noticeable shift of any peak that appeared in the spectra of the gelatin or PVA scaffolds was detected in the spectra of the composite scaffolds. For instance, the amide I band of both the 1:1 and 1:3 PVA/gelatin core-shell scaffolds was at 1656 cm⁻¹, indicating an α-helix structure. Therefore, the FTIR analysis indicates no alteration in the secondary structures of the gelatin shell during the coaxial electrospinning process.

### Mechanical Analysis

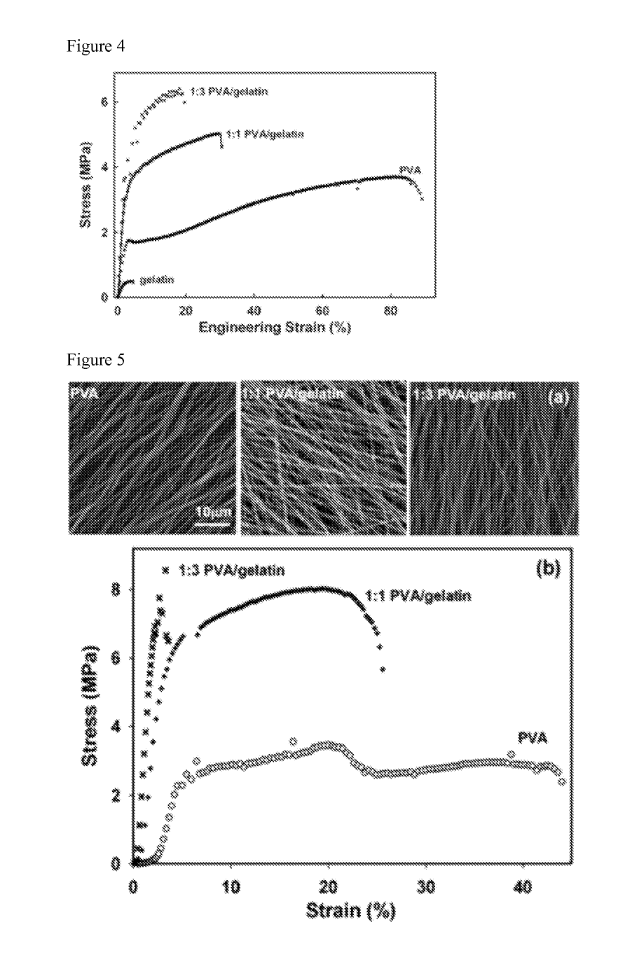

The randomly oriented fibers of crosslinked gelatin, PVA, 1:1 PVA/gelatin composite, and 1:3 PVA/gelatin composite scaffolds were tested in a uniaxial tensile mode to generate stress-strain curves (FIG. 4). The Young's modulus, ultimate strength, and strain at failure were evaluated from the stress-strain curves and recorded in Table II for each of the scaffold materials. The gelatin scaffolds possessed the lowest Young's modulus (21.5 6 4.2 MPa), ultimate tensile strength (0.48±0.02 MPa), and strain at failure (4.3±0.5%). These results were comparable with the GTA-crosslinked gelatin scaffolds reported by Zha et al. (Biopolymers 2012, 97, 1026-1036). The low strain at failure of the gelatin scaffolds is attributed to their low water content. Water functions as a plasticizer, increasing the deformability of protein-based materials (Zha et al., Biopolymers 2012, 97, 1026-1036; Qiu et al., Biomacromolecules 2009, 10, 602-608). When compared with the gelatin scaffolds, the PVA scaffolds had higher strain at failure (88.2±39.8%), Young's modulus (100.5±23.5 MPa), and ultimate tensile strength (3.92±1.19 MPa). It was anticipated that the PVA core would enhance the mechanical properties of the composite scaffolds more than the gelatin-only nanofiber scaffolds.

Indeed, both the 1:1 PVA/gelatin and 1:3 PVA/gelatin composite nanofiber scaffolds possessed a higher Young's modulus, a higher ultimate strength, and larger strain at failure than the gelatin scaffolds. For instance, the Young's modulus and ultimate tensile strength of the 1:1 PVA/gelatin composite scaffolds are 7.8 and 11.3 times higher than the gelatin scaffolds, respectively. A close analysis of the mechanical properties of the coreshell composite scaffolds further reveals that the PVA/gelatin composite scaffolds are stronger but less deformable than the PVA scaffolds. When compared with the PVA scaffolds, the 1:1 PVA/gelatin composite scaffolds displayed a nearly 70% increase in Young's modulus (168.6±36.5 MPa vs. 100.65±23.5 MPa), a 40% increase in ultimate tensile strength (5.42±1.95 MPa vs. 3.92±1.19 MPa), and a 60% reduction in strain at failure (88.2±39.8% vs. 37.1±14.4%). Second, the 1:3 PVA/gelatin composite scaffolds contain less PVA but are mechanically stronger than the 1:1 PVA/gelatin scaffolds (221.5±28.4 MPa vs. 168.6±36.5 MPa for Young's modulus and 6.59±0.45 MPa vs. 5.42±1.95 MPa for tensile strength). The comparisons indicate that the presence of the gelatin shell improves the mechanical properties of the PVA core.

It is postulated that the gelatin shell enhances the molecular alignment of PVA molecules and thus mechanically strengthens the PVA core. In electrospinning, charges tend to accumulate on the surface of the solution jet, leading to the occurrence of various instabilities, including, in particular, the Rayleigh instability (Hohman, M. M. et al., Phys Fluids 2001, 13, 2201-2220; Hohman, M. M. et al., Phys Fluids 2001, 13, 2221-2236). During the process of coaxial electrospinning, the elasticity of the shell fluid can delay or even suppress the Rayleigh instability of the core fluid (Sun, Z. C. et al., Adv Mater 2003, 15, 1929-1936). Consequently, the semicrystalline PVA core is stretched, molecularly aligned, and crystallized more often. This, in turn, would transform the plastic PVA core into a more elastic PVA core. The mechanical properties of the core-shell composite scaffolds are thus improved, leading to an increased Young's modulus, a higher tensile strength, and reduced plastic deformation.

To further investigate this, aligned nanofibers of PVA, 1:1 PVA/gelatin, and 1:3 PVA/gelatin were fabricated (FIG. 5a), crosslinked, and tested in tension as previously described (Table III). A similar trend in the stress-strain was observed with the aligned nanofibers as was seen with the randomly oriented nanofibers (FIG. 5b). The 1:1 PVA/gelatin composite nanofibers had a 29.5% increase in Young's modulus when compared with aligned PVA nanofibers (254.9±42.4 MPa vs. 196.9±57.7 MPa). Additionally, the 1:3 PVA/gelatin aligned nanofibers possessed a 19.6% increase in Young's modulus (304.9±86.7 MPa vs. 254.9±42.4 MPa) even though the 1:3 PVA/gelatin nanofibers contain less PVA than the 1:1 PVA/gelatin aligned nanofibers. This provides further indication of increased molecular alignment of PVA chains within the composite nanofibers.

### Cell Adhesion

To be used for biomedical applications, electrospun scaffolds should be bioactive in that they promote cellular adhesion, proliferation, differentiation, and tissue regeneration (Liu et al., J Biomed Mater Res Part A 2005, 74, 84-91). PVA is devoid of functional groups that promote cellular attachment and proliferation. In a study by Nien et al. (J Med Biol Eng 2009, 29, 98-101) fibroblasts cultured on a PVA scaffold showed a round morphology instead of a native spindle-like shape. In contrast to PVA, gelatin is composed of high molecular weight polypeptides derived from collagen (Nien et al., J Med Biol Eng 2009, 29, 98-101; Liu, X. et al., J Biomed Mater Res Part A 2005, 74, 84-91; Lin, Y. et al., Acta Biomater 2006, 2, 155-164). The triple helix structure of collagen is broken down into a single-stranded structure to form gelatin, which is rich in cellular attachment groups for high bioactivity (Liu, X. et al., J Biomed Mater Res Part A 2005, 74, 84-91; Lin, Y. et al., Acta Biomater 2006, 2, 155-164; Bhardwaj, N. et al., Biotechnol Adv 2010, 28, 325-347). Therefore, it is contemplated that the presence of gelatin in the shell of the core-shell composite nanofibers produces nanofibers with biological properties similar to gelatin and better than PVA alone.

In this study, the tissue culture polystyrene plate (TCP) served as the negative control, and the absorbance of the adhered fibroblasts on the scaffold was normalized by that of fibroblasts adhered on the TCP (FIG. 6). Among all the samples, the gelatin nanofibers had the greatest ability to support cellular adhesion (113.1±5.2%), which was attributed to the numerous cellular attachment sites of gelatin. Because PVA lacks cellular recognition sites, the PVA nanofibers possessed the lowest rate of cellular adhesion (63.9±4.9%) when compared with the gelatin and coaxial scaffolds (P<0.001 for PVA vs. gelatin; P<0.01 for PVA vs. 1:1 PVA/gelatin; P<0.03 for 1:3 PVA/gelatin). The composite nanofibers placed gelatin in the shell position with PVA in the core position, thereby exposing the highly bioactive gelatin and shielding PVA from the cellular environment. The combination of these materials in the core-shell structure uses gelatin in a way that maximizes its biological properties. The cellular adhesion of the composite nanofibers possessed statistically similar results to each other (1:1 PVA/gelatin 101.8±9.7%; 1:3 PVA/gelatin 93.6±9.6%; P 5 0.57) and to gelatin (113.1±5.2%; P=0.35 for gelatin vs. 1:1 PVA/gelatin; P=0.13 for gelatin vs. 1:3 PVA/gelatin). Therefore, the gelatin shell was capable of increasing the adhesion of fibroblasts to the core-shell nanofibers in comparison with the PVA nanofiber scaffolds. Additionally, the PVA/gelatin composite nanofibers were free of deficiencies, minimizing any compromise of cellular adhesion that was observed in other beaded nanofiber scaffolds (Yang, C. et al., J Appl Polym Sci 2011, 121, 3047-3055).

### Cell Proliferation

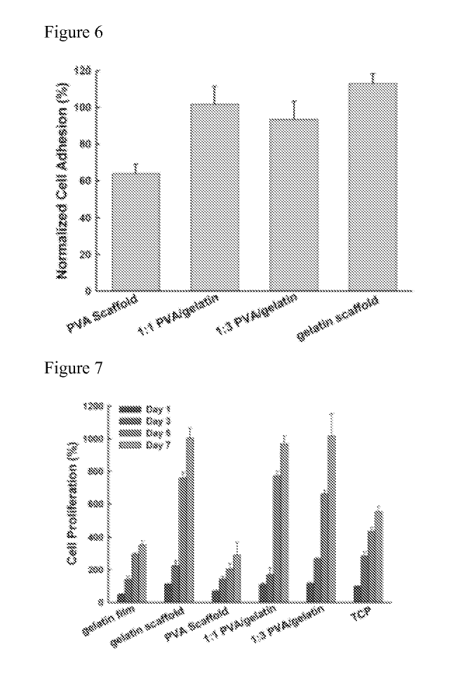

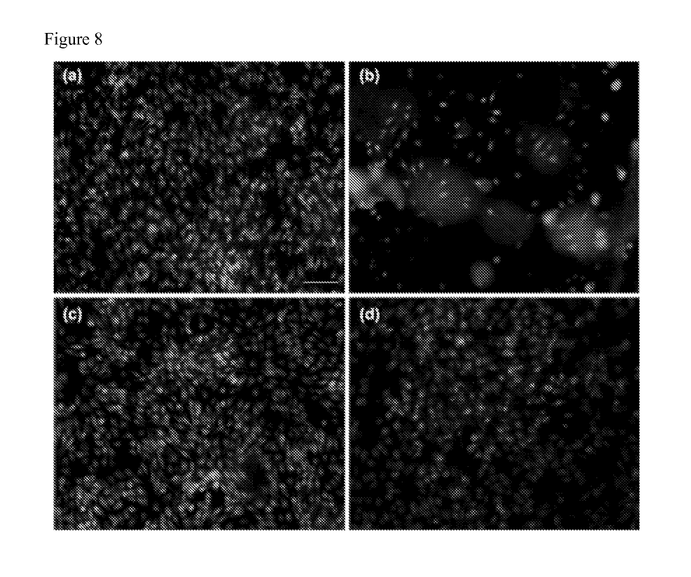

A MTT assay was used to assess the ability of the gelatin, PVA, and 1:1 PVA/gelatin and 1:3 PVA/gelatin core-shell scaffolds to support cellular proliferation. The TCP and gelatin films were used as the control. All scaffolds were seeded with the same cellular density. The assay uses a reduction reaction of yellow MTT to purple formazan in the mitochondria of living cells. The quantity of formazan present is directly related to the number of viable cells. Assays were completed at day 1, day 3, day 5, and day 7 after initial cell seeding. The absorbance of each sample over all time points was normalized to the absorbance from the TCP at day 1. During the 7 days of cell culture, all the scaffolds as well as the TCP showed a continual increase in cell number (FIG. 7). The TCP and the PVA scaffolds showed a linear increase in the proliferation of 3T3 fibroblasts over the 7 days of cell culture. The gelatin, 1:1 PVA/gelatin, and 1:3 PVA/gelatin composite nanofibers showed a dramatic increase in cellular proliferation from day 3 to day 5. This large increase was not present for the gelatin film. Therefore, the gelatin nanofibers promoted more cellular growth than the two-dimensional films that possessed cellular proliferation similar to the PVA scaffolds. Furthermore, the gelatin, 1:1 PVA/gelatin, and 1:3 PVA/gelatin composite nanofiber scaffolds displayed statistically similar cellular proliferation rates (P=0.66 for 1:1 PVA/gelatin vs. gelatin; P=0.93 for 1:3 PVA/gelatin vs. gelatin; P=0.75 for 1:1 PVA/gelatin vs. 1:3 PVA/gelatin); these scaffolds statistically supported more proliferation of fibroblasts than the PVA scaffold (P<0.0003 for gelatin vs. PVA; P<0.0003 for 1:1 PVA/gelatin vs. PVA; P<0.002 for 1:3 PVA/gelatin vs. PVA) or the TCP (P<0.0003 for gelatin vs. TCP; P<0.0006 for 1:1 PVA/gelatin vs. TCP; P<0.016 for 1:3 PVA/gelatin vs. TCP). Therefore, this study confirmed that the presence of gelatin in the shell position of the core-shell nanofibers maximized the biological properties of gelatin and that the three dimensional scaffolds possessed a greater ability to support cell proliferation than the two-dimensional flat surfaces.

### Cell Viability

In vitro cell viability and cyto-compatibility of the scaffolds were analyzed through a NIH/3T3 fibroblast model. Using the live/dead assay to determine viability, living cells emitted a green fluorescence due to the calcein AM that crossed the cellular membrane of live cells. Dead cells emitted a red fluorescence due to the penetration of the EthD-1 (ethidium homodimer-1) dye through the damaged cellular membrane. Once inside the cell, it bonded to the nucleic acids, producing the red fluorescence. The viability assay showed that most of the cells seeded on the gelatin, PVA, 1:1 PVA/gelatin, and 1:3 PVA/gelatin nanofiber scaffolds were alive (FIG. 8). Cell viability was quantified by counting the number of live cells versus the total cells on each type of scaffold. The percent viability of 3T3 fibroblasts on the gelatin, PVA, 1:1 PVA/gelatin, and 1:3 PVA/gelatin nanofiber scaffolds was determined to be 95.03±0.86, 92.66±7.02, 99.12±0.51, and 98.94±0.93%, respectively. When compared with the gelatin and PVA/gelatin coreshell nanofiber scaffolds, 3T3 fibroblasts on the PVA scaffold displayed a comparable percent viability. At the same initial cell seeding density, however, much fewer cells grew on the PVA scaffold than other scaffolds. This is consistent with the proliferation study (FIG. 7). Together with the cell adhesion and proliferation analyses, the viability assay indicated that the cytobiocompatibility of the 1:1 and 1:3 PVA/gelatin nanofiber scaffolds is comparable with that of the gelatin scaffold.

In conclusion, this example demonstrates that composite nanofibers fabricated with gelatin in the fiber shell and PVA in the fiber core that are much stronger than the gelatin scaffolds. When compared with the PVA scaffolds, the PVA/gelatin core-shell nanofiber scaffolds also display enhanced mechanical properties, including an increased Young's modulus, a higher tensile strength, and reduced plastic deformation. Increasing the gelatin/PVA mass ratio from 1:1 to 1:3 further improved the mechanical properties of the composite scaffolds. A cell culture study further showed that cellular adhesion and proliferation on the core-shell nanofiber scaffolds were statistically similar to the gelatin scaffolds and much better than the PVA scaffolds. Therefore, the placement of gelatin in the fibers' shells maximized the biological properties of gelatin. Taken together, coaxial electrospinning as a fiber-formation technique allows the fabrication of PVA/gelatin composite nanofibers with a core-shell structure that fully uses the bioactivity of gelatin and the mechanical property of PVA.

TABLE I
Fiber Diameter and Pore Size of Nanofiber Scaffolds

| | Fiber Diameter (nm) | Pore Size (μm) |
|---|---|---|
| Gelatin | 223 ± 93 | 1.55 ± 0.49 |
| PVA | 283 ± 188 | 1.65 ± 0.71 |
| 1:1 PVA/gelatin | 256 ± 99 | 1.46 ± 0.70 |
| 1:3 PVA/gelatin | 182 ± 81 | 2.15 ± 0.59 |

TABLE II
Mechancal Properties of Randomly Oriented Nanofibers

| | Young's Modulus | Ultimate | Strain at |
|---|---|---|---|
| | (MPa) | Strength (MPa) | Failure (%) |
| Gelatin | 21.5 ± 4.2 | 0.48 ± 0.02 | 4.3 ± 0.5 |
| PVA | 100.5 ± 23.5 | 3.92 ± 1.19 | 88.2 ± 39.8 |
| 1:1 PVA/gelatin | 168.6 ± 36.5 | 5.42 ± 1.95 | 37.4 ± 14.4 |
| 1:3 PVA/gelatin | 221.5 ± 28.4 | 6.59 ± 0.45 | 21.4 ± 8.1 |

TABLE III
Mechanical Properties of Aligned Nanofibers

| | Young's Modulus | Ultimate | Strain at |
|---|---|---|---|
| | (MPa) | Strength (MPa) | Failure (%) |
| PVA | 196.9 ± 57.7 | 4.17 ± 0.39 | 36.7 ± 14.8 |
| 1:1 PVA/gelatin | 254.9 ± 42.4 | 13.16 ± 6.12 | 21.7 ± 13.7 |
| 1:3 PVA/gelatin | 304.9 ± 86.7 | 7.42 ± 1.13 | 2.8 ± 0.8 |

### Example 2

### Methods

### Nanofiber Fabrication

A 16% w/v solution of PVA was prepared in a mixture of ethanol and deionized water (1:9 v/v) at 60° C., and type A gelatin was dissolved in a binary solvent of ethanol and 10× phosphate buffered saline (PBS) at a 1:1 volume ratio at a concentration of 15% w/v at 40° C. A coaxial apparatus containing an inner needle of 0.25 mm in diameter and an outer needle of 0.84 mm in diameter was used to fabricate PVA/gelatin and gelatin/PVA core shell nanofibers. The two solutions were delivered at a feeding rate of 7 μL/min (Kazel Syringe Pumps), leading to a roughly 1:1 mass ratio of PVA and gelatin in the composite nanofibers. A spinneret-to-collector distance of 15 cm and a high voltage of 20 kV (Acopian High Voltage Power Supply) were utilized. A custom-built electrospinning device was used to fabricate PVA and gelatin nanofibers (Qiu W G, et al., Biomacromolecules 2010; 11:3219; Qiu W G, et al., Appl Phys Lett 2011; 98:263702). The PVA solution was placed in a 1 mL syringe with a 0.25 mm inner diameter needle, and electrospun at a voltage of 12 kV, a spinneret-to-collector distance of 12 cm, and a feeding rate of 9 μL/min. The gelatin solution was placed in a 1 mL syringe with a 0.33 mm inner diameter needle, and electrospun at a voltage of 12 kV, a spinneret-to-collector distance of 12 cm, and a feeding rate of 30 μL/min.

Aligned nanofibers were electrospun using a gap method (Li D, et al., Nano Lett 2003; 3:1167). Briefly, a stainless steel plate with a rectangular-shaped hole cut in the center was used as the grounded collector. The same electrospinning parameters and solutions were used, as described for the randomly aligned nanofibers. Aligned nanofibers were deposited bridging the gap on the stainless steel plate.

### Nanofiber Characterization

All the nanofibers were crosslinked using a 0.5% glutaraldehyde solution, and dried in a vacuum desiccator for 20 h at 40° C. prior to mechanical and secondary structural analysis, following a previously described protocol (Qiu W G, et al., Biomacromolecules 2010; 11:3219; Qiu W G, et al., Appl Phys Lett 2011; 98:263702). An optical microscope was utilized to determine the original thickness of rectangular strips of each nanofibrous scaffold (Qiu W G, et al., Biomacromolecules 2010). Uniaxial tensile testing was then completed in an ambient environment using a PerkinElmer dynamic mechanical analyzer. Five replicate samples of each scaffold with a gauge length of 8 mm were monotonically extended at a rate of 0.1 mm/min until failure. A Nicolett Magna IR 560 spectrometer equipped with an MCT/A detector recorded the FT-IR spectra of the nanofiber scaffolds. Samples were prepared on zinc-selenium windows. FT-IR spectra were obtained within the range of 4000-650 cm⁻¹ using 128 scans with a corresponding resolution of 2 cm⁻¹. The surface morphologies of the nanofiber scaffolds were studied using a Hitachi-S4800 field emission SEM. Prior to microscope observation, the scaffolds were air dried for 24 h, mounted, and coated with gold for 60 s in a sputter coater. Image J was used to calculate the average fiber diameter and pore size from SEM images of each scaffold type (Qiu W G, et al., Appl Phys Lett 2011; 98:263702). The FEI CM128 TEM, operated at 80 kV, was utilized to obtain images of the core-shell structure of the PVA/gelatin nanofibers that were collected on a copper grid prior to imaging. The porosity of the electrospun scaffolds was calculated by determining the apparent density of each scaffold and comparing it to the polymer density using the following equation (Vaz C M, et al., Baaijens F P T. Acta Biomater 2005:575):

where ρ is the density of the electrospun scaffold and ρ₀ is the density of the bulk polymer.

### Results

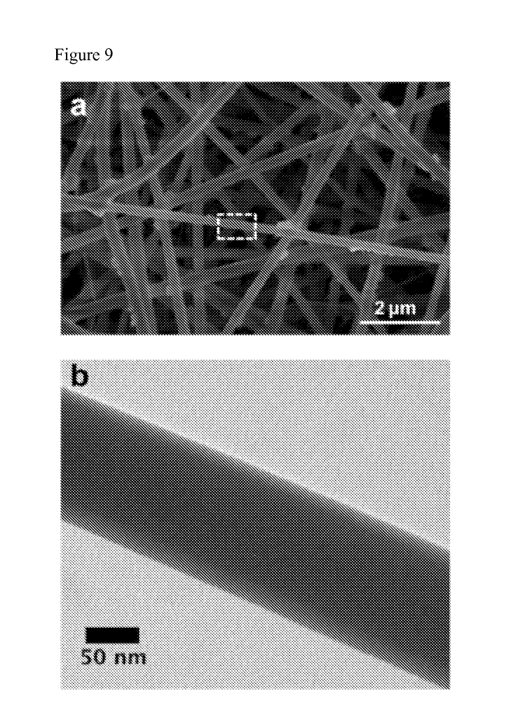

As shown in FIG. 9a, relatively uniform, bead-free PVA/gelatin coreshell nanofibers of 255±99 nm were generated via coaxial electrospinning Filaments of less than 50 nm in diameter were also formed, due to the emanation of lateral branches from an unstable solution jet in the electrospinning process. Additionally, coreshell nanofibrous scaffolds possessed an average pore size of 1.46±0.70 mm. Due to the use of PBS as a solvent, nanometer-sized salt crystals appear on the surfaces of the composite nanofibers. Previously, gelatin nanofibers were electrospun from cytotoxic solvents (Kim H W, et al., Adv Funct Mater 2005; 15:1988; Huang Z M, et al., Polymer 2004; 45:5361) or corrosive solvents (Gu S Y, et al. Mater Sci Eng C Mater Biol Appl 2009; 29: 1822; Song J H, et al., J Mater Sci Mater Med 2008; 19:95; Park Y H, et al., Um I C. Polymer 2005; 46:5094; Yang M C, et al., Polym Adv Technol 2009; 20:98). The incomplete removal of these solvents may compromise the long-term biocompatibility of the gelatin nanofibrous scaffolds. To avoid the use of organic solvents, the electrospinning of gelatin in aqueous solutions at elevated temperatures was pursued (Zhang S, et al., J Biomed Mater Res Part A 2009; 90:671). In this study, PBS was used instead of water as a solvent for gelatin to coaxially electrospin PVA/gelatin nanofibers, because ions such as K⁺ and Na⁺ from PBS tend to break down the gelatin network and prevent the gelation of gelatin solutions. The addition of ethanol disrupts hydrogen bonding between gelatin and water molecules, enhances intermolecular interactions between gelatin segments of complementary charges, and increases the rates of solvent removal from the solution jet. As a result, the binary solvents of ethanol and PBS with a proper balance of ionic strength and ethanol content provide a benign system for the electrospinning of gelatin solutions at room temperature. Salts accumulated on the PVA/gelatin composite nanofibers can be readily removed by washing the scaffolds in water.

The formation of a coreshell structure in the PVA/gelatin nanofibers was confirmed by TEM (FIG. 9b). It is worthwhile noting that PVA solutions can be blended with gelatin or collagen solutions for the fabrication of fibers or films (Yang D Z, et al., J. Carbohydr Polym 2007; 69:538; Sionkowska A, et al., J. Polym Degrad Stab 2009; 94:383). However, the short flight time of the solution jets largely limits the diffusion driven mixing of PVA and gelatin solutions during electrospinning (Sun Z C, et al., Adv Mater 2003; 15: 1929). As a result, coreshell nanofibers are generated, in which PVA forms the cores, providing mechanical strength, while gelatin constitutes the shells, rendering the composite nanofibers biologically active.

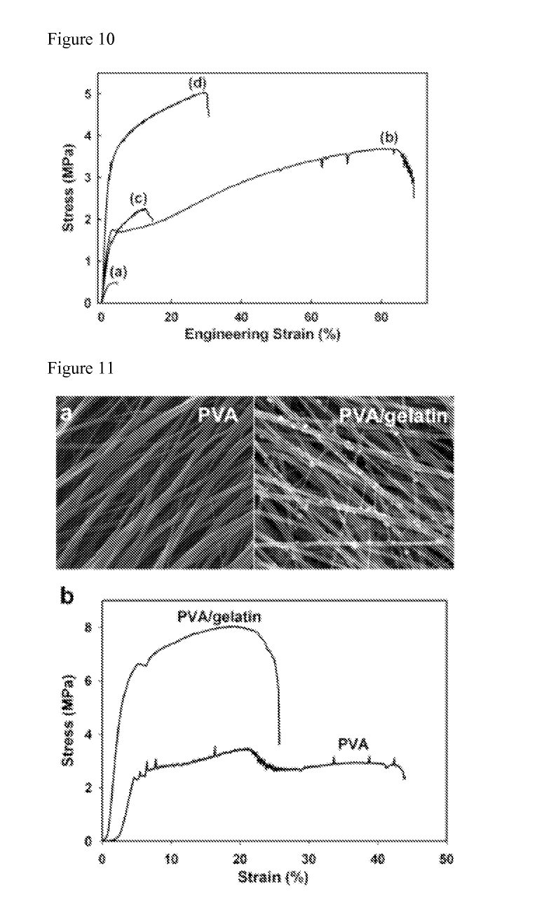

Mechanical analysis of PVA/gelatin coreshell composite scaffolds reveals a Young's modulus (E) of 168.6±36.5 MPa, ultimate tensile strength (σf) of 5.42±1.95 MPa, and strain at failure (εf) of 37.4±14.4% (FIG. 10). As a control, PVA nanofiber scaffolds possess E of 100.5±23.5 MPa, σf of 3.92±1.19 MPa, and εf of 88.2±39.8%. Their mechanical properties are consistent with those of the PVA scaffolds electrospun from pure aqueous solutions (Jeong J S, et al., Thin Solid Films 2007; 515:5136). Gelatin nanofibrous scaffolds display an E of 21.52±4.15 MPa, a σf of 0.48±0.02 MPa, and an εf of 4.3±0.5%, which are comparable to the scaffolds electrospun from aqueous solutions at elevated temperature (Zhang S, et al., J Biomed Mater Res Part A 2009; 90:671) but inferior to the scaffolds electrospun from trifluoroethanol (Zhang Y Z, et al., Polymer 2006; 47: 2911). Compared to the PVA scaffolds, the PVA/gelatin coreeshell scaffolds display a 68% increase in Young's modulus, a 38% increase in tensile strength, and a nearly 60% reduction in strain at failure.

To ensure that the mechanical properties are not attributed to differences in porosity between the coreshell nanofibers and the gelatin and PVA scaffolds respectively, the average fiber diameter, pore size and porosity are determined. The coreshell nanofibers possess an average fiber diameter of 256±99 nm, in comparison to gelatin and PVA which possess an average diameter of 223±93 nm and 283±188 nm, respectively. Furthermore, the average pore size of the coreshell nanofibers is 1.46±0.70 mm, which is similar to gelatin nanofibers (1.55±0.49 mm) and PVA nanofibers (1.65±0.71 mm). Lastly, the average porosity of the coreshell nanofiber is 76.4±16.3%, which is similar to gelatin nanofibers (74.8±9.9%) but slightly higher than PVA nanofibers (60.8±7.2%). In comparison to PVA, therefore, the porosity of the coreshell nanofibers is not responsible for the mechanical strengthening effects.

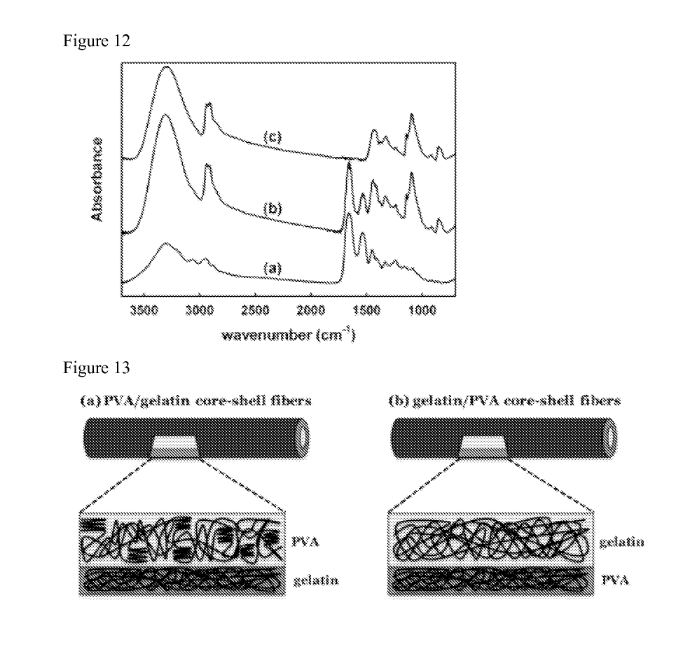

Aligned coreshell nanofibers were also fabricated and mechanically analyzed as an additional method to illustrate the mechanical strengthening observed with PVA (FIG. 11). Aligned nanofibers of PVA were electrospun for comparison (FIG. 11a). Coreshell nanofibers possess an E of 235.50 MPa±33.28, a σf of 13.16±6.12 MPa, and an εf of 24.85±8.22%. In comparison, PVA scaffolds possess an E of 163.55±65.57 MPa, a σf of 3.48±1.32 MPa, and an εf of 45.31±12.70 MPa. Similar trends in the mechanical properties are observed for both the randomly oriented nanofibers and the aligned nanofibers. The present invention is not limited to a particular mechanism. Indeed, an understanding of the mechanism is not necessary to practice the invention. Nonetheless, several possible mechanisms may be responsible for the mechanical strengthening effects observed in the PVA/gelatin coreshell nanofiber scaffolds. They include (i) the enhanced alignment of PVA molecules by the gelatin shell, (ii) the improved alignment of gelatin molecules by the PVA core, and (iii) interactions between PVA and gelatin molecules. FT-IR spectroscopy of the PVA, gelatin, and PVA/gelatin coreshell nanofibers was performed to determine the underlying mechanism of the mechanical strengthening effects (FIG. 12). The PVA nanofibers display characteristic double bands at 2937 and 2908 cm⁻¹ (CH₂ and CH stretching), 1143 and 1194 cm⁻¹ (CC skeletal stretching), and single bands at 1426 cm⁻¹ (CH₂ and CH scissoring/wagging) and 852 cm⁻¹ (CC skeletal stretching); the gelatin nanofibers exhibit distinct amide I and II bands at 1658 and 1536 cm⁻¹, respectively. The PVA/gelatin coreshell nanofibers display all the characteristic bands of the PVA and gelatin nanofibers at the same positions. The lack of any shift of the existing bands and the formation of new bands indicates no strong interactions between the gelatin shell and the PVA core. Further, the amide I bands of the gelatin and PVA/gelatin coreshell nanofibers, which are sensitive to the configurations of protein backbones and often used for a quantitative analysis of the secondary structures of a protein (Teng W B, et al. J Phys Chem B 2011; 115:1608; Taddei P, et al., Biopolymers 2005; 78:249), have the same peak and shape. This indicates that gelatin molecules in the gelatin and PVA/gelatin coreshell nanofibers possess the same configurations of protein backbones. Therefore, the FT-IR analysis indicates no appreciable enhancement in the alignments of gelatin molecules by the PVA core or strong interactions between PVA and gelatin molecules that may strengthen the composite nanofibers.

It is thus postulated that the gelatin shell improves the alignment of PVA molecules during coaxial electrospinning and enhances the formation of hydrogen bonds between the hydroxyl groups (OH) and the crystallinity of PVA in the core (FIG. 13a). In the FT-IR spectrum of the PVA nanofibers, a broad band at 3301 cm⁻¹ is ascribed to the stretching of both free and hydrogen-bonded OH groups. An enhanced crystallinity of the PVA cores leads to a shift of the amide A to a higher frequency. However, the gelatin nanofibers also display a very broad band at 3310 cm⁻¹, making any slight shift of the amide A band of the PVA core difficult to detect. An enhanced crystallinity of the PVA core, nevertheless, would result in an increase in Young's modulus and tensile strength but a reduction in strain at failure. This is consistent with the mechanical analysis of the PVA/gelatin coreeshell scaffolds (FIGS. 10 and 11).

Therefore, it is hypothesized that the elasticity of the gelatin shell delays or suppresses the Rayleigh instability of the PVA core, enhancing the molecular alignment of PVA. Specifically, charges accumulate on the surface of the solution jet during electrospinning, and instability in various forms occurs on the surface (Hohman M M, et al., Phys Fluids 2001; 13:2201; Hohman M M, et al., Phys Fluids 2001; 13:2221). During coaxial electrospinning, the gelatin shell solution shields the PVA core solution from the turbulent surface, allowing the PVA molecules to be more stretched and better aligned. As a result, semi-crystalline, plastic PVA in the core is partially transformed into more crystallized, elastic PVA, and the mechanical properties of the PVA/gelatin coreshell nanofibers are thus improved.

To test this, reverse coreshell nanofibers with gelatin in the core and PVA in the shell were fabricated and mechanically analyzed. In the reverse coreshell nanofibers, no enhanced alignment of PVA molecules is expected (FIG. 13b). Like PVA, physical treatments such as stretching may improve the crystallinity and mechanical properties of gelatin (Bigi A, et al., Biomaterials 1998; 19:2335). However, the gelatin nanofibers electrospun from aqueous solution are much weaker than the PVA nanofibers. Therefore, even if the PVA shell may strengthen the gelatin core, the mechanical properties of the gelatin/PVA coreshell nanofibers will be inferior to the PVA/gelatin coreshell nanofibers. Indeed, the reverse coreshell nanofibers possess an E of 91.38±2.88 MPa, a σf of 3.10±0.76 MPa, and an εf of 20.18±10.23% (FIG. 10). Together with FT-IR spectroscopy, tensile analysis confirms that the mechanically weak gelatin shell improves the molecular alignment and mechanical properties of the PVA core of the composite nanofibers.

In conclusion, PVA and gelatin were coaxially electrospun into composite nanofibers, in which PVA forms the core and gelatin constitutes the shell. The coreshell nanofibers display an increased Young's modulus, improved tensile strength, and reduced plastic deformation over the PVA nanofibers. FT-IR spectroscopy showed neither enhanced alignment of gelatin molecules in the shell nor strong interactions between the PVA core and the gelatin shell. The gelatin shell improves the molecular alignment of the PVA core, transforming semi-crystalline, plastic PVA into more-crystallized, stronger, elastic PVA and thereby mechanically strengthening the core. This was confirmed by reversing the order of PVA and gelatin in the coreshell nanofibers and by analyzing the mechanical properties of the

### Example 3

### Materials & Methods

### Electrospinning

A 16% w/v solution of PVA (Sigma Aldrich, 89,000-98,000 MW, 99+% hydrolyzed) was prepared with ethanol and water (1:9 v/v) in a water bath for 4 hours at 60° C. Electrospun PVA nanofibers were fabricated from a custom-built electrospinning device using the following parameters: 12 kV applied high voltage (Acopian High Voltage Power Supply), 12 cm fixed needle-tip to collector distance, and 9 μL/min fixed flow rate (Razel Syringe Pump, R-99). A 15% w/v solution of Gelatin Type A (Sigma Aldrich) was prepared from ethanol and 10λ-phosphate buffered saline (1:1 v/v) in a water bath for 4 hours at 40° C. Gelatin scaffolds were fabricated using the following parameters: 12 kV applied voltage, 12 cm fixed needle-tip to collector distance, and a 30 μL/min flow rate. The coaxial nanofibers were fabricated using a custom-coaxial electrospinning device using the previously described gelatin and PVA solutions. Composite fibers were made using two different flow rate conditions: 3 Gelatin: 1 PVA (3 μL/min flow rate for PVA and 9 μL/min flow rate for gelatin) and 1 gelatin: 1 PVA (7 μL/min for gelatin and PVA, respectively). Each composite scaffold used a 20 kV applied voltage and 15 cm needle-tip to collector distance. All scaffolds were crosslinked with 5% glutaraldehyde in ethanol in a vacuumed dessicator for 20 hours at 42° C.

Cell Culture Electrospun scaffolds were placed in a vacuum oven for 24 hours at 42° C. to remove residual glutaraldehyde. Subsequently, scaffolds were sterilized in the ultraviolet light for 30 minutes before placing in a 24-well tissue culture plate. Scaffolds were incubated for 15 minutes with approximately 150 μL of cell media prior to cell seeding. After incubation with the cell culture media, the stem cells were added and the final media volume per well was 500 μL. The cells were seeded on the scaffolds and incubated at 37° C., 5% CO₂, and 95% relative humidity. Cell culture media was refreshed every 2 days. As a control, cells were seeded on the tissue culture polystyrene well (TCP) without any ECM present.

Cell culture media for the adipose-derived stem cells was MEM alpha mod. (lx) stock supplemented with 10% fetal bovine serum, 1% L-glutamine, 1% non-essential amino acids, 1% pen/strep, and 1% sodium pyruvate. ASC were grown to 80% confluency and passaged using trypsin-versene.

### Cell Morphology & Spreading

Approximately 30,000 adipose-derived stem cells were seeded on the scaffold or tissue culture plate surface and incubated at 37° C., 5% CO₂, and 95% relative humidity for 3, 5, and 7 days. After incubation, stem cells were fixed for scanning electron microscopy (SEM) using 5% glutaraldehyde. The cells/scaffolds were then transferred from glutaraldehyde to de-ionized water through a series of graded solutions. Next, solutions were transferred to ethanol using a series of graded solutions and allowed to remain in the ethanol overnight. Finally, the samples were transferred to hexamethyldisilazane (HMDS) through a series of graded solutions and air dried overnight (Thomasson S A, et al., Transaction of the Kansas Academy of Science 2011; 114:129-34). Samples were gold-coated for 60 seconds and imaged using a Hitachi-S4800 SEM. Cell spreading was calculated from the SEM images using the National Institute of Health's ImageJ software.

### Cellular Viability

Cellular viability was assessed using a lactate dehydrogenase (LDH) assay (Thermo Scientific) according to manufacturer's instructions. The LDH assay was completed 1, 3, 5, and 7 days after initial cell seeding.

### Cellular Adhesion

Scaffolds were electrospun onto stainless steel chips/squares and crosslinked as described in the previous section. Stem cells (75,000 cells/well) were seeded on the scaffold and allowed to incubate for 4 hours. After the 4 hours of incubation, the scaffolds were rinsed four times with 1× phosphate buffered saline (PBS). Next, cell quantity remaining on the scaffolds was assessed using a MTT (thiazolyl blue tetrazolium bromide) assay. First, 100 μL of MTT solution (5 mg/mL of MTT in 1×PBS) was added to each well and incubated for 3.5 hours. After the 3.5 hours of incubation, the media/MTT solution was aspirated and dimethyl sulfoxide (DMSO) added. The absorbance of the resulting purple solution was read using a spectrophotometer at 630 nm.

### Cellular Proliferation

Cellular proliferation on the electrospun scaffolds and tissue-culture plate was assessed using a MTT (thiazolyl blue tetrazolium bromide) assay. Scaffolds and the TCP were seeded with 7,500 cells/well and the proliferation assay was completed at days 1, 3, 5, and 7 after initial cell seeding. The media was refreshed every 2 days. The MTT assay was completed at each desired time point as discussed in the previous section. Additionally, scaffolds were fixed and stained at each time point to assess confluency on the scaffold surface. Stem cells were rinsed with 1× phosphate buffered saline (PBS) and fixed with SafeFix II (Fisher Diagnostics, Middletown, Va., USA) for 10 minutes. After cell fixing, cells were washed twice with PBS-T (1×PBS with 1% of Tween 20—Sigma Aldrich, St. Louis, Mich., USA) to permeabilize the cells and stained with 0.1% toluidine blue (Sigma Aldrich, St. Louis, Mich., USA) for 10 minutes.

### Cellular Migration

Cellular migration was completed using a non-injury cylinder assay. Scaffolds were sterilized in ultraviolet light and incubated for 15 minutes with 10 μl of cell culture media. Stem cells were diluted to 125,000 cells/mL and 40 μl of the cell suspension was added to the center of a hollow cylinder (Pyrex® cloning cylinders—Fisher Scientific, Pittsburgh, Pa. USA) resting on the scaffold or culture plate surface of a 24-well plate (adding 5,000 stem cells per scaffold). The cells and scaffolds were incubated for 4 hours to allow the cells to seed on the scaffold surface in the center of the hollow cylinder. After the 4 hours of incubation, the cylinders were removed, and 500 μl of stem cell media was added.

Stem cells were allowed to migrate on the respective surface for 0, 4, 24, or 48 hours. After stem cell migration, scaffolds/cells were rinsed with 1× phosphate buffered saline (PBS) and fixed with SafeFix II (Fisher Diagnostics, Middletown, Va., USA) for 10 minutes. After cell fixing, cells were washed twice with PBS-T (1×PBS with 1% of Tween 20—Sigma Aldrich, St. Louis, Mich., USA) and stained with 0.1% toluidine blue (Sigma Aldrich, St. Louis, Mich., USA) for 10 minutes.

The cellular migration assay was repeated using basic fibroblast growth factor (bFGF) at a concentration of 20 ng/ml. In this assay, stem cells (40 μl of 125,000 cells/mL) were seeded into the center of a hollow cylinder and incubated for 4 hours. After the 4 hours of incubation, the cylinder was removed, cell culture media added (460 μl), and bFGF added. Stem cells were allowed to migrate for 0, 4, 24, or 48 hours. Cells were fixed as previously described.

### Statistical Analysis

All values are presented as the mean±standard error of the mean unless otherwise indicated. Statistical analysis was performed using the student's t-test.

### Results

### Cellular Morphology & Viability

Coaxial electrospun nanofibers were fabricated with gelatin in the shell and polyvinyl alcohol (PVA) in the core of each nanofiber. Therefore, these coaxial fibers have the structural benefit of PVA in the core and the biological benefit of gelatin in the shell. In this study, coaxial nanofibers with the following mass ratios were fabricated: 1 Gel:1 PVA and 3 Gel:1 PVA. The increase in the mass ratio of gelatin present in the coaxial nanofibers created a thicker gelatin sheath on the fibers as was previously reported (Merkle V, et al., Polymers 2013; Merkle V M, et al., Biopolymers 2013). For a material to be utilized as a tissue-engineered construct, cellular response to the scaffold, namely morphology, viability, adhesion, proliferation, and migration should be assessed.

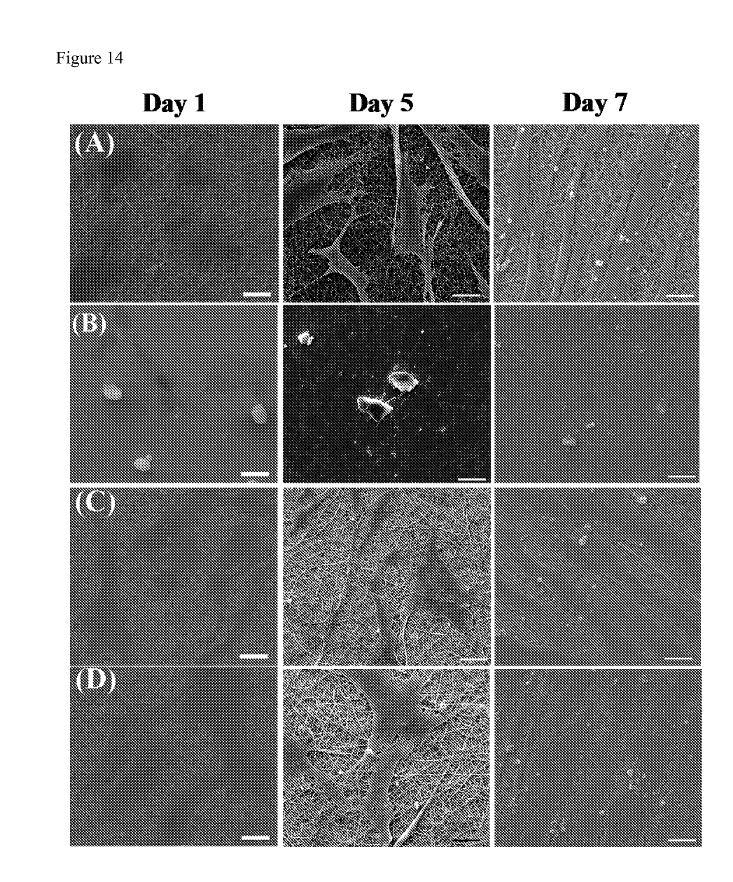

In order to determine cell morphology on the electrospun scaffolds, adipose-derived stem cells (ADSC) were seeded on each scaffold, incubated for 24 hours, and prepared for SEM imaging. The stem cells displayed a flattened morphology with multiple attachment sites to the underlying fibers on the gelatin and coaxial scaffolds (1 Gel:1 PVA and 3 Gel:1 PVA) (FIG. 14). Gelatin is a natural polymer derived from the collagen triple helix; therefore, this polymer has multiple cell recognition sites (Sisson K, et al., Biomacromolecules 2009; 10:1675-80; Zhang Y Z, et al., Biomacromolecules 2005; 6:2583-9; Zhao P, et al., Journal of Biomedical Materials Research Part A 2007:372-82). The PVA scaffolds showed stem cells that were spread on the nanofibers with multiple attachment sites, as well as multiple cells with a round morphology with minimal attachment sites. This is in contrast to fibroblast morphology seen on the synthetic, hydrophilic PVA scaffold where the cells presented only in a round morphology with minimal attachment sites (Nien Y H, et al., Journal of Medical and Biological Engineering 2009; 29:98-101; Merkle V, et al., Polymers 2013; Schmedlen R, et al., Biomaterials 2002; 23:4325-32).

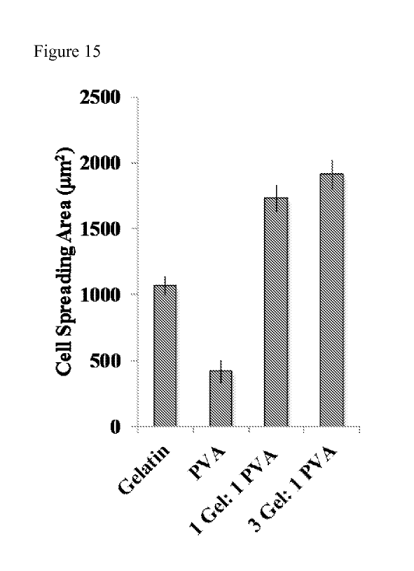

ADSC spreading on each of the electrospun scaffolds was calculated from the SEM images taken at 24 hours of incubation (Table 4 & FIG. 15). The PVA scaffolds had the lowest cell spreading of all of the scaffolds with an average cell area of 421.1±85.4 μm² (p<0.00001). Next, the gelatin scaffolds had a cell spreading of 1068.8±69.5 μm². The cell spreading then increased for the 1 Gel:1 PVA coaxial scaffolds (1731.5±104.8 μm²) and the 3 Gel:1 PVA coaxial scaffolds (1911.6±114.4 μm²), which had similar cell spreading (p=0.25) and the highest cell spreading area when compared with gelatin scaffolds (p<0.00001) or PVA scaffolds (p<0.00001). This increase in cell spreading observed on the gelatin and coaxial scaffolds correlated with the increase in stiffness observed with each of these scaffolds. The gelatin scaffolds had the lowest Young's modulus (21.5±4.2 MPa). The stiffness increased with the 1 Gel:1 PVA coaxial scaffold (100.5±23.5 MPa), and the 3 Gel:1 PVA coaxial scaffold had the highest Young's modulus (221.5±28.4 MPa) (Merkle V, et al., Polymers 2013; Merkle et al., Biopolymers 2013).

TABLE 4
Cell Spreading

| | Cell Area |
|---|---|
| | (μm²) |
| Gelatin | 1068.8 ± 69.5 |
| PVA | 421.1 ± 85.4 |
| 1 Gel: 1 PVA Coaxial | 1731.5 ± 104.8 |
| 3 Gel: 1 PVA Coaxial | 1911.6 ± 114.4 |

Cellular morphology was also observed after 5 and 7 days of incubation (FIG. 14). After 1 day of incubation on the scaffolds, the ADSC had a flattened morphology with multiple attachment sites on the gelatin and coaxial scaffolds. Additionally, the cells on the gelatin and coaxial scaffolds had no preferential alignment. After 5 days of incubation, the cells started to elongate on the gelatin and coaxial scaffolds; however, there was still cells present with a wider, spread morphology. After 7 days of cell culture, the ADSC possessed an elongated morphology with the cells aligned along the long cellular axis. In contrast, the PVA scaffolds had a mix of round and flattened morphology after 24 hours in culture. By 5 and 7 days in culture, the ADSC on the PVA scaffolds were predominantly in the round morphology.

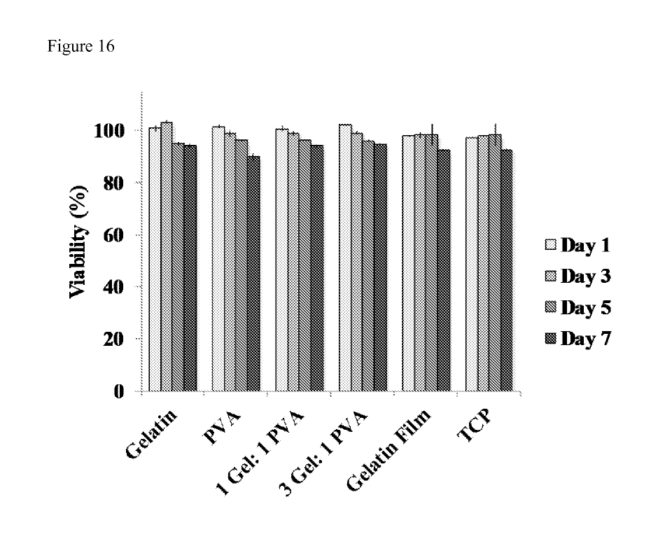

Next, cellular viability was assessed using the lactate dehydrogenase (LDH) assay. LDH seeps through the cell membrane of damaged cells where it allows the conversion of pyruvate to lactate using the reduction of NAD⁺ to NADH. With NADH now present, the tetrazolium salt is converted to red formazan through the diaphorase enzyme. The absorbance of the red formazan is then read through a plate reader to determine cellular viability. Cellular viability was determined at days 1, 3, 5, and 7 after initial cell seeding (FIG. 16). At each time point, all of the scaffolds, tissue culture plate, and gelatin film had a cellular viability greater than 90%.

### Cellular Adhesion

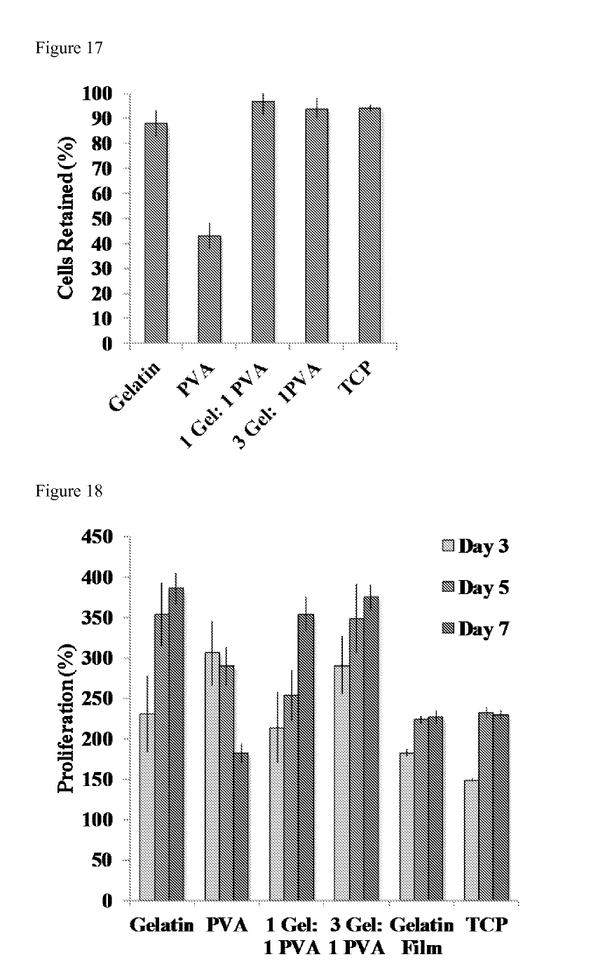

After obtaining high cellular viability throughout the 7 days in culture, cellular adhesion on the electrospun scaffolds was then determined. First, ADSC were seeded on the electrospun scaffolds for 4 hours and subsequently rinsed several times with 1×PBS to remove any cells not adherent to the fibers or tissue culture plate surface. Cell quantity was then determined using a MTT assay (FIG. 17). The gelatin and the coaxial scaffolds (1 Gel:1 PVA coaxial and 3 Gel:1 PVA coaxial) had similar ADSC adhesion (p>0.34). The PVA scaffolds had the lowest cell adhesion with 43.1±5.3% cells retained (p<0.001).

### Cellular Proliferation

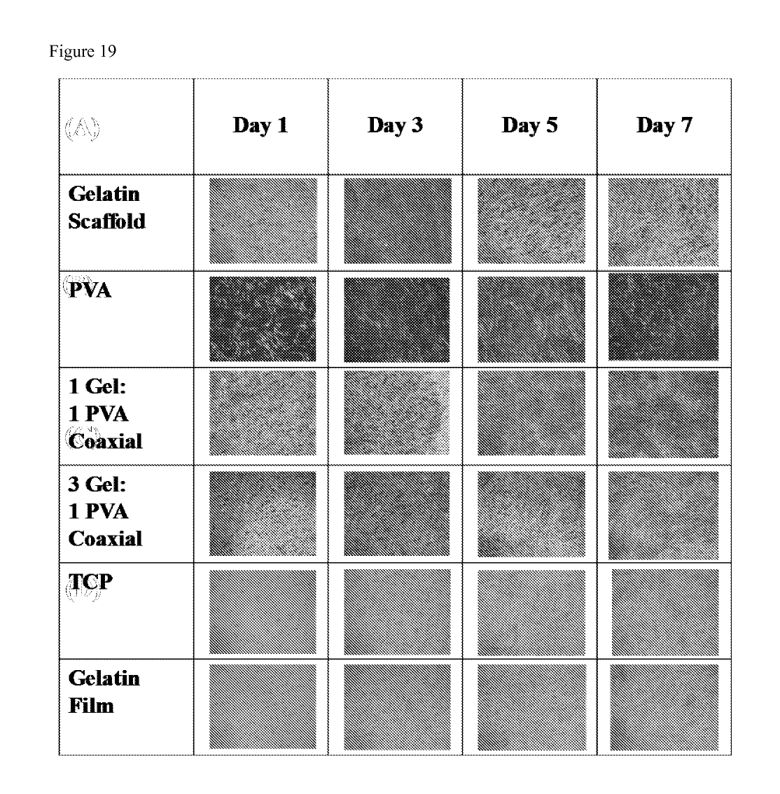

ADSC proliferation was assessed at days 3, 5, and 7 after cell seeding on the gelatin scaffold, PVA scaffold, 1 Gel:1 PVA coaxial scaffold, 3 Gel:1 PVA coaxial scaffold, gelatin film, and tissue culture plate (FIG. 18). Overall, the gelatin scaffold, coaxial scaffolds (1 Gel:1 PVA coaxial and 3 Gel:1 PVA coaxial), gelatin film, and tissue culture plate (TCP) had continued cell growth until reaching confluency over 7 days in culture (FIG. 19). The gelatin scaffolds (385.4±19.2%) and coaxial scaffolds (1 Gel:1 PVA coaxial (353.3±21.2%), and 3 Gel:1 PVA coaxial (374.8±15.6%)) promoted very high ADSC proliferation after 7 days in culture compared to the gelatin film (226.9±8.0%) and tissue culture plate (229.4±5.3%). Overall, the gelatin film promoted higher cell growth than the TCP on day 3 (p<0.007); however, the surfaces reached confluence and maintained similar cell quantities by days 5 and 7 (p=0.80) (FIG. 19). The higher cell growth seen on the gelatin scaffolds compared to the gelatin films indicate that the scaffold structure promotes higher cell proliferation than the flat, film surface of the same material (p<0.00001). At all time points, the gelatin film and TCP had lower cell numbers than the gelatin scaffolds, 1 Gel:1 PVA coaxial scaffolds, and 3 Gel:1 PVA coaxial scaffolds.

The PVA scaffolds started with a high cell growth on day 1; however, there was a steep decline in cell count throughout the 7 days of cell culture. While the scaffolds didn't show any cytotoxicity, the PVA scaffolds did not support a continual increase in cell quantity over 7 days of incubation. On day 3, the PVA scaffolds had cell growth approximately 305.8±40.1%, which was still higher than the TCP. By day 7 however, the cell count had decline to approximately 182.0±12.5%. This observation can be attributed to the synthetic nature of the PVA polymer itself. PVA lacks cellular recognition sites that promote cellular adhesion, as well as increased cell proliferation.

### Cellular Migration

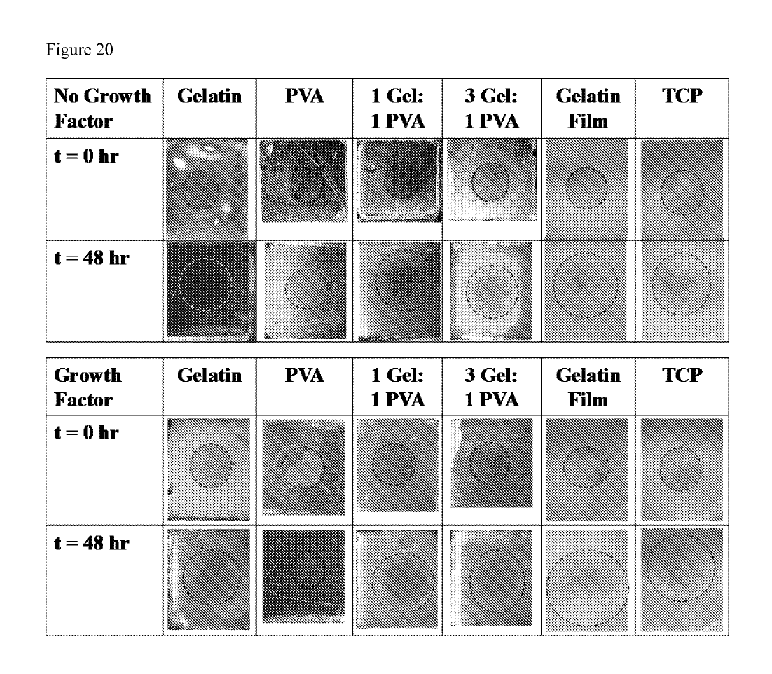

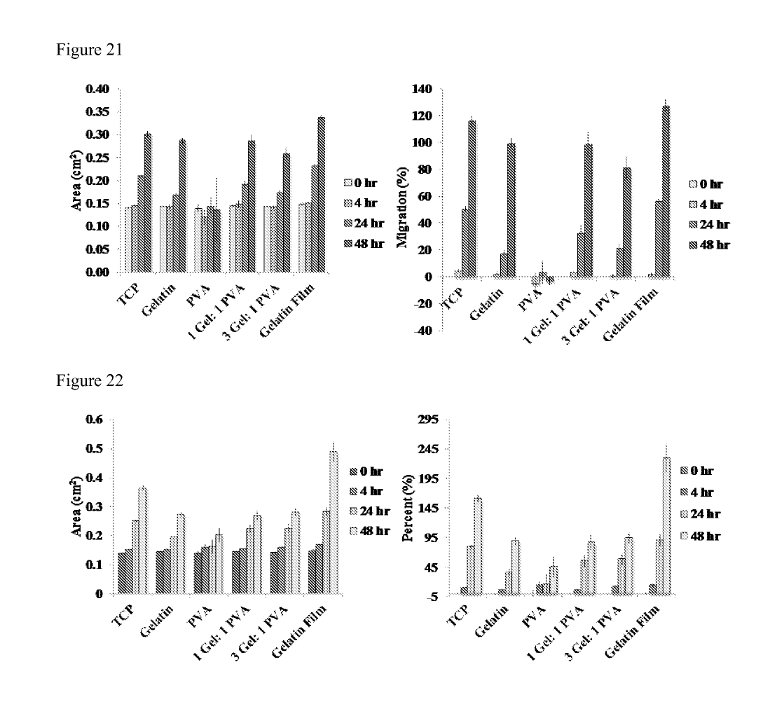

ADSC migration was assessed using a non-injury cylinder assay in which the cells were seeded into the center of a hollow cylinder and allowed to migrate outward for 0, 4, 24, or 48 hours. After this migration time, the ASC were fixed, imaged, and the percent outward migration calculated (FIG. 20). The gelatin scaffold, 1 Gel:1 PVA coaxial scaffold, 3 Gel:1 PVA coaxial scaffold, TCP, and gelatin film showed continued outward cell migration after 48 hours. Overall, the gelatin film had the highest outward cellular migration over the 48 hours when compared to gelatin scaffold (p<0.0005), PVA scaffold (p<0.0001), 1 Gel:1 PVA coaxial scaffold (p=0.01) and 3 Gel:1 PVA coaxial scaffold (p<0.0001) (FIG. 21). The higher migration seen on the gelatin film rather than the gelatin scaffold indicates that the scaffold structure, although promoting high cellular proliferation, does not enhance cellular migration. Additionally, the gelatin film and TCP had similar high outward migration after the 48 hours of incubation (p=0.07).

Basic fibroblast growth factor (bFGF), a pro-migratory growth factor, was added to the cell culture media to determine the effects of ADSC migration on the film and scaffold surfaces (FIG. 22). Basic fibroblast growth factor, also known as FGF-2 or b-FGF, belongs to a family of growth factors that are involved in wound healing, as well as angiogenesis (Rodrigues M, et al., Stem Cell Research and Therapy 2010; 1). In a study completed by Schmidt et al., bFGF was shown to increase ADSC migration in a dose dependent manner until a migratory maximum was reached. This growth factor was shown to increase migration 2.3× the migration seen in the control using a modified Boyden chamber (Schmidt A, et al., Stem Cells 2006; 24:1750-8). When implanted into the body, the scaffold and cells come into contact with growth factors, such as bFGF that can affect migration. Therefore, any potential increases that may occur when the scaffolds are in a pro-migratory environment was assayed.

Overall, the gelatin film had the highest outward migration after 48 hours when in the presence of the growth factor when compared to the TCP (p<0.02), gelatin scaffold (p<0.0002), PVA scaffold (p<0.0001), 1 Gel:1 PVA coaxial scaffold (p<0.0004), and 3 Gel:1 PVA coaxial scaffold (p<0.0004) (FIG. 22). Next to the gelatin film, the TCP had the next highest outward migration. The PVA had the lowest outward cellular migration with little, if any, cellular migration. Overall, the gelatin scaffold, 1 Gel:1 PVA coaxial scaffold, and 3 Gel:1 PVA coaxial scaffold had similar migration with the growth factor present as was seen without the growth factor present. Therefore, even with the growth factor present, the scaffold structure did not enhance the outward migration of ADSC when compared to TCP or gelatin film.

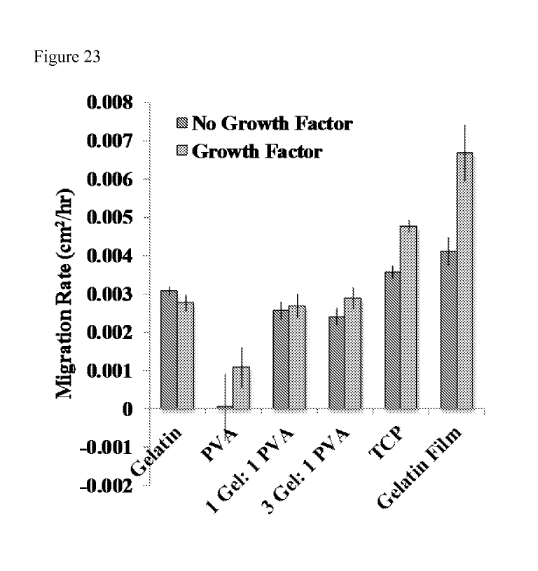

The migration rates were calculated over the 48 hours for the outward migration experiments with and without the bFGF present to determine the percent change per hour in cell area for each scaffold, the TCP, and the gelatin film (FIG. 23). With the growth factor present, the gelatin film (p<0.02) and the TCP (p<0.01) had increases in migration rate. However, the gelatin scaffold (p=0.27), PVA scaffold (p=0.42), 1 Gel:1 PVA coaxial scaffold (p=0.75), and 3 Gel:1 PVA coaxial scaffold (p=0.21) had similar rates of migration with and without the growth factor present. Therefore, even in a pro-migratory environment, ADSC do not have an increase in cell migration on scaffold surfaces.

Biocompatible scaffolds that promote the viability and growth of adipose-derived stem cells are important for tissue engineering and regenerative medicine applications. All of the electrospun scaffolds (gelatin, PVA, 1 Gel:1 PVA coaxial, and 3 Gel:1 PVA coaxial), as well as the tissue culture plate (TCP) and gelatin film supported ADSC viability over 7 days of culture. Scanning electron microscopy (SEM) showed flattened spread morphology with multiple attachment sites on the gelatin and coaxial scaffolds with the coaxial scaffolds having the largest spread area. The PVA scaffolds had the lowest spread area with mixed morphology of flattened spread morphology, as well as a round morphology after 24 hours of incubation. For cellular adhesion, the PVA scaffolds had the lowest cell adhesion due to the lack of cellular attachment sites on this synthetic polymer. The scaffold structure promoted very high cellular proliferation after 1 day in culture in comparison to the gelatin film and TCP. For cellular migration, the gelatin film and TCP had higher outward migration than the electrospun scaffolds. The outward migration rates on the electrospun scaffolds were similar with and without a pro-migratory growth factor present. Overall, the electrospun scaffolds (gelatin, 1 Gel:1 PVA coaxial, and 3 Gel:1 PVA coaxial) provides a topographic substrate similar to the native extracellular matrix promoting the viability and growth of ADSC while entrapping and affixing them for maturation and differentiation. These electrospun nanofibers provide a platform for use in tissue engineering and regenerative medicine applications.

### Example 4

### Methods

Electrospinning.

A 16% w/v solution of PVA (Sigma-Aldrich, 89 000-98 000 MW, 99+% hydrolyzed) was prepared with ethanol and water (1:9 v/v) in a water bath for 4 h at 60° C. The use of ethanol and water as solvents increased the evaporability of solvents for subsequent electrospinning compared to using only water as a solvent. Electrospun PVA nanofibers were fabricated from a custom built electrospinning device using the following parameters: 12 kV applied high voltage (Acopian High Voltage Power Supply), 12 cm fixed needle-tip to collector distance, and 9 μL/min fixed flow rate (Razel Syringe Pump, R-99). A 15% w/v solution of Gelatin Type A (Sigma-Aldrich) was prepared from ethanol and 10×-phosphate buffered saline (PBS) (1:1 v/v) in a water bath for 2 h at 40° C. and subsequently at room temperature for 12 h for further dissolution. Gelatin nanofibers were fabricated using the following parameters: 12 kV applied voltage, 12 cm fixed needle-tip-to-collector distance, and a 30 μL/min flow rate. The coaxial nanofibers were fabricated using a custom-coaxial electrospinning device using the previously described gelatin and PVA solutions. Composite fibers were made using two different flow rate conditions: 3 Gel:1 PVA (3 μL/min flow rate for PVA and 9 μL/min flow rate for gelatin) and 1 Gel:1 PVA (7 μL/min for gelatin and PVA, respectively). Each composite scaffold used a 20 kV applied voltage and 15 cm needle-tip-to-collector distance. After electrospinning was complete, all scaffolds were then cross-linked using a glutaraldehyde/ethanol vapor. To cross-link the scaffolds, a 5% glutaraldehyde in ethanol solution was prepared and placed in the bottom of a desiccator. Next, the scaffolds were placed on the desiccator shelf and vacuum was pulled. The desiccator was then placed in an oven at 42° C. for 20 h. After the 20 h in the oven, the scaffolds were ready for subsequent experiments as described in the proceeding sections.

Fiber Surface Roughness.

The samples were gold coated under argon gas at 70 mTorr for 60 s. The samples were imaged in a Bruker dimensional atomic force microscopy (AFM) using tapping mode. The surface roughness (Ra) was determined using the Bruker Nanoscope Analysis v1 0.40r2 software for 25 fibers of each scaffold (gelatin, PVA, 1 Gel:1 PVA coaxial, and 3 Gel:1 PVA coaxial). The images were corrected using the 3rd order-flattening tool to remove tilts and bows from the images. The roughness (Ra) values were obtained using the roughness tool with the peak inputs on. The peak inputs allowed one to define a threshold height from the surface to measure the roughness. This ensured that only the roughness of the fiber was obtained.

Blood Samples.

Blood samples were taken from healthy adults who signed informed consent forms. Adults did not take aspirin or ibuprofen for 2 weeks prior to donating and had not consumed caffeine for 12 h prior to blood draw. Thirty milliliters of whole blood was drawn via venipuncture and added to 0.3 mL 40% trisodium citrate. The whole blood was centrifuged at 450 g for 4.5 min. The platelet-rich plasma (PRP) was removed from the sample. The PRP was filtered through a 150 mL column of Sepharose 2B beads (2% agarose; Amersham-Pharmacia, Sigma Chemical, St. Louis, Mo.) to yield the gel-filtered platelets (GFP).

Platelet Viability.

Platelet viability was assessed using a lactate dehydrogenase (LDH) assay (Thermo Scientific) after 3 h of incubation on the electrospun scaffolds or tissue culture plate (TCP) according to manufacturer's instructions.

Platelet Deposition on Electrospun Scaffolds.

Five hundred microliters of 20,000 platelets/μL was incubated on the scaffold or substrate surface. Platelets and scaffolds were incubated at 37° C., 5% CO₂, and 95% relative humidity for 4 h. After incubation, platelets were fixed for scanning electron microscopy (SEM) using 5% glutaraldehyde. The cells/scaffolds were then transferred from glutaraldehyde (GTA) to deionized water through a series of graded solutions (3 GTA:1 H₂O, 1 GTA:1 H₂O, and 1 GTA:3 H₂O). Next, solutions were transferred to ethanol (EtOH) using a series of graded solutions (3 H₂O:1 EtOH, 1 H₂O:1 EtOH, and 1 H₂O:3 EtOH) and allowed to sit overnight in the ethanol solution. Lastly, the samples were transferred to hexamethyldisilazane (HMDS) through a series of graded solutions (3 EtOH:1 HMDS, 1 EtOH:1 HMDS, and 1 EtOH:3 HMDS) and then allowed to air-dry overnight (Thomasson, S. A. et al., Trans. Kans. Acad. Sci. 2011, 114, 129-134). After preparation, the samples were gold-coated for 60 s using a sputter coater and imaged using field emission scanning electron microscopy (SEM, Hitachi-S4800). Platelet deposition on scaffolds was determined using the National Institute of Health's ImageJ software using ten images for each scaffold type.

For the mechanically activated platelets, platelets at a concentration of 20,000 platelets/μL were subjected to a physiological shear of 10 dyne/cm2 for 10 min using the hemodynamic shearing device (Nobili, M. et al., ASAIO J. 2008, 54, 64-72). Platelets were then seeded onto the scaffolds and incubated for 4 h as described in the preceding paragraph. After the 4 h of incubation, the scaffolds were prepared for SEM imaging as previously described. For the chemically activated platelets, platelets at a concentration of 20,000 platelets/μL and 5 μM adenosine diphosphate (ADP) were incubated on each scaffold for 4 h. Samples were prepared for SEM imaging as described previously.

Platelet Activation Assay.

Platelet activation state (PAS) was measured using a chemically modified prothrombinase-based assay. Each scaffold was seeded with 500 μL of human platelets at a concentration of 20,000 platelets/μL in platelet buffer. Samples were incubated at 37° C. for 0, 60, 120, and 180 min. After incubation, 200 nM Factor IIa (acetylated prothrombin), 100 pM factor Xa, and 5 mM calcium (Ca2+) were added and incubated for 10 min. After the 10 min incubation, samples were read at 405 nm for 7 min in a plate reader at 405 nm to obtain the PAS values. The PAS values at each time point were normalized against fully activated platelets (obtained using 7.5 W of sonication for 10 s). The normalized value represents the fraction of thrombin that is produced by fully sonicated platelets. The platelet activation rate (PAR) is obtained from the slope of the PAS values over 3 h (Jesty, J. et al., Anal. Biochem. 1999, 272, 64-70; Sheriff, J. et al., Ann. Biomed. Eng. 2010, 38, 1442-1450; Nobili, M. et al., ASAIO J. 2008, 54, 64-72).

Platelet Deposition Under Flow Conditions.

Platelet deposition on the electrospun scaffolds was determined under vascular relevant shear conditions to better simulate the in vivo environment. Platelet-rich plasma was diluted to a concentration of 15 000 platelets/μL and circulated for 60 min across the scaffold surfaces at 1 or 3 dyn/cm2, respectively. The samples were then fixed and imaged using a SEM in order to determine platelet deposition.

Effects of SMC or HUVEC Preseeding on Platelet Deposition.

To prepare the scaffolds for cell culture, the scaffolds were placed in an oven at 42° C. for a period of 24 h. Next, the scaffolds were sterilized in the ultraviolet light for 30 min prior to cell culture. The scaffolds were then incubated with cell culture media for a period of 15 min prior to cellular seeding. Cell culture media for human umbilical vein endothelial cells (HUVEC) was M-199 stock media supplemented with 1% 0.2 M glutamine, 1.5% 1 M HEPES (Lonza Walkersville, Walkersville, Md., USA), 7.5% NaHCO3, 1.8% penicillin/streptomycin/gentamicin (Lonza Walkersville, Walkersville, Md., USA), 15% fetal calf serum (Lonza Walkersville, Walkersville, Md., USA), heparin salt (Fisher Bioreagants, Fair Lawn, N.J., USA) and ECGS (endothelial cell growth supplements). Human umbilical vein endothelial cell (HUVEC) (BD Biosciences, San Jose, Calif., USA) were grown to 80% confluency and were passaged using a 1:1 mixture of trypsin-versene and HBSS (Lonza Walkersville, Md., USA).

Cell culture media for the primary smooth muscle cells (SMC) was Dulbecco's modified Eagle's medium (Life Technologies, Carlsbad, Calif., USA) supplemented with 10% v/v fetal calf serum (Life Technologies, Carlsbad, Calif., USA), antibiotic/antimycotic (Life Technologies, Carlsbad, Calif., USA), and 1% 0.2 M L-glutamine (Lonza Walkersville, Walkersville, Md., USA). The primary smooth muscle cells were isolated from the aorta of an adult, male Sprague Dawley rat. SMC were also grown to 80% or higher confluency and passaged using trypsinversene.

After incubation with the cell culture media, the respective cell line was seeded on the scaffold and incubated at 37° C., 5% CO₂, and 95% relative humidity for 3 days with the media refreshed on day 2. As a control, cells were seeded on the tissue culture polystyrene well (TCP) without any scaffolds present, as well as a gelatin film coated on the tissue culture polystyrene well. After the 3 days of incubation, the scaffolds were rinsed three times with 1× phosphate-buffered saline (PBS) and once with platelet buffer After rinsing with platelet buffer, the scaffolds were seeded with platelets at a concentration of 20,000 platelets/μL for 4 h. After incubation, platelet deposition on the cell-coated scaffolds was determined as previously described.

Statistical Analysis.

All results are presented as mean±standard error unless otherwise indicated. Statistical analysis was completed using a two-tailed, unpaired student's t test.

### Results

Fiber Surface Roughness.

For a material to be a successful vascular construct, the material should be evaluated to ensure low platelet activation while maintaining the platelet's role in hemostasis and angiogenesis (Rubenstein, D. A. et al., J. Biomater. Sci., Polym. Ed. 2010, 21, 1713-1736). Enhanced surface roughness increases platelet adhesion and the presence of platelet pseudopodia to the underlying substrate. Therefore, there is an increase in platelet adhesion, spreading, and subsequent platelet activation (Milleret, V. et al., Acta Biomater. 2012, 8, 4349-4356; Fatisson, J. et al., J. R. Soc., Interface 2011, 8, 1-10). In this experiment, the fiber surface roughness of the gelatin, PVA, and coaxial nanofibers (1 Gel:1 PVA coaxial and 3 Gel:1 PVA coaxial) was determined because this parameter plays a role in platelet adhesion and subsequent activation.

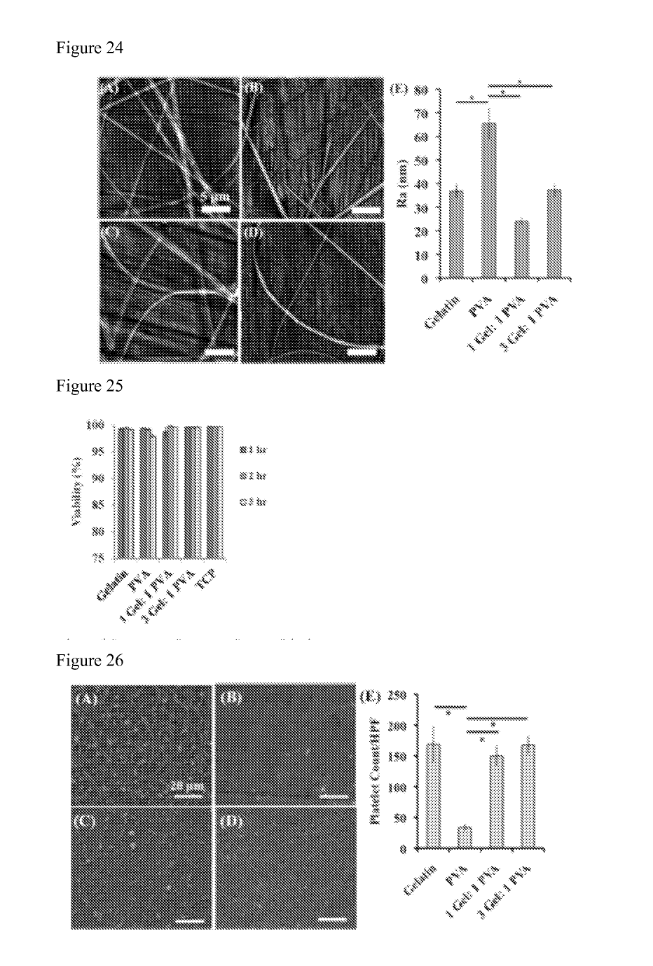

A Bruker atomic force microscope and the Bruker Nanoscope Analysis software were utilized to determine the surface roughness of individual nanofibers (FIG. 24). The fiber surface roughness is reported as Ra, representing the average surface height deviations from a given reference point (e.g., the surface of the stainless steel chip that the fibers are electrospun onto).

The use of the reference point allows one to determine the surface roughness of each individual fiber without taking into account the stainless steel substrate that the fibers were electrospun on. Out of all of the fibers, PVA had the highest surface roughness (Ra=65.5±6.8 nm) when compared to gelatin (p<0.001), 1 Gel:1 PVA coaxial scaffolds (p<0.001), and 3 Gel:1 PVA coaxial scaffolds (p<0.001). This led to an investigation as to if the increased surface roughness of the PVA nanofibers lead to an increase in platelet deposition and activation in comparison to the less rough fibers of gelatin and the coaxial nanofibers. These fiber surface roughness values were used with the proceeding analyses for platelet deposition and activation.

Platelet Viability on Electrospun Nanofibers.

Platelet viability was determined using a lactate dehydrogenase (LDH) assay (Naghadeh, H. T. et al., Blood Transfus. 2013, 11, 400-404; Gupta, A. et al. J. Transfus. Sci. 2011, 5, 160-163). Damage to the cell membrane causes lactate dehydrogenase to leak into the platelet buffer. The LDH now in the media can then convert the pyruvate to lactate through the reduction of NAD+ to NADH. With NADH now present, the diaphorase can then convert tetrazolium salt to red formazan, which can then be quantified using a spectrophotometer at a wavelength of 490 nm. All scaffolds (gelatin, PVA, 1 Gel:1 PVA coaxial, and 3 Gel:1 PVA coaxial) and the tissue culture polystyrene plate (TCP) possessed platelet viability of 98+% at hours 1, 2, and 3 of incubation (FIG. 25). Therefore, the scaffold surfaces promote platelet viability throughout all subsequent platelet deposition and activation studies.

Platelet Deposition on Electrospun Nanofibers Under Static Conditions.

Platelet deposition after incubation was calculated using SEM images (3000× magnification, approximately 8400 μm²) and the National Institute of Health's ImageJ software (FIG. 26). The gelatin scaffolds (169±30 platelets) and the coaxial scaffolds (1 Gel:1 PVA coaxial, 150±17 platelets; 3 Gel:1 PVA coaxial scaffold, 168±15 platelets) possessed similar platelet deposition and significantly more platelets than the PVA scaffolds (34±6 platelets). Despite the PVA fibers' having the highest surface roughness, these fibers had the lowest platelet deposition out of all of the scaffolds. Gelatin, rich in cellular attachment sites, has higher platelet deposition than the PVA fibers with significantly higher surface roughness and lacking cellular attachment sites. Therefore, surface roughness was not a dominating factor in platelet deposition. The chemical signals present on the biocompatible gelatin fibers were a dominant factor in platelet deposition in comparison to the synthetic polymer PVA, lacking these cellular recognition sites.

Platelet Activation on Electrospun Scaffolds Under Static Conditions.

In addition to platelet deposition, platelet activation is an important determinant of a material's hemocompatibility and potential to serve as a vascular construct. Activated platelets in the presence of factor II (prothrombin) and factor Xa form a prothrombinase complex that catalyzes the conversion of prothrombin to thrombin. In the normal coagulation cascade, thrombin formation provides a positive feedback on the activation of platelets and additional thrombin formation (Jesty, J. et al., Anal. Biochem. 1999, 272, 64-70). Therefore, quantification of platelet activation using thrombin can be difficult. In this example, a modified prothrombinase assay in which acetylated prothrombin is utilized was performed. Essentially, this assay determines the quantity of acetylated thrombin formed from acetylated prothrombin using low platelet concentrations (20,000 platelets/μL). Use of the acetylated prothrombin at low platelet concentrations inhibits the positive feedback of the thrombin formation on the platelets. Thus, a 1:1 correlation is achieved between platelet activation and acetylated thrombin formation (Jesty, J. et al., Anal. Biochem. 1999, 272, 64-70).

Electrospun scaffolds were incubated with 5000 μL of platelets at a concentration of 20,000 platelets/μL for 0, 1, 2, or 3 h. If the platelets are activated from the interaction with the nanofibers, then the anionic phospholipid (phosphatidylserine) is translocated from the inner leaflet to the outer leaflet of the cell membrane. Phosphatidylserine will then bind and activate coagulation factors VII, IX, X, and prothrombin. Additionally, activated platelets will activate factor V, present in the α-granules, and express it on the membrane surface. Activated factor V is required for the factor Xa activation of prothrombin. Therefore, activated platelets produce the necessary cofactors needed for the formation of acetylated prothrombin, which is measured in subsequent steps of this assay (Jesty, J. et al., Anal. Biochem. 1999, 272, 64-70). At each time point (t=0, 60, 120, or 180 min), acetylated factor II, factor Xa, and Ca2+ were added to the well and incubated at 37° C. for 10 min to start the assay. After the 10 min incubation, a chromogenic substrate (Chromozyme-TH) for thrombin is added, which allows the amount of acetylated thrombin to be quantified in a plate reader at 405 nm for 7 min. The slope of the absorbance over the 7 min reading is referred to as platelet activation state (PAS). The PAS values are normalized with the PAS value obtained for fully activated platelets. Therefore, the normalized PAS value represents the fraction of acetylated thrombin formed from fully activated platelets. The platelet activation rate (PAR) is the slope of the individual PAS values at each time point throughout the modified prothrombinase assay (0, 1, 2, and 3 h), which indicates the overall rate of change of platelet activation. Therefore, the PAR indicates how rapidly the platelets are becoming activated when in the presence of the electrospun scaffolds. Overall, the PAR is used for comparison of platelet activation between the scaffolds.

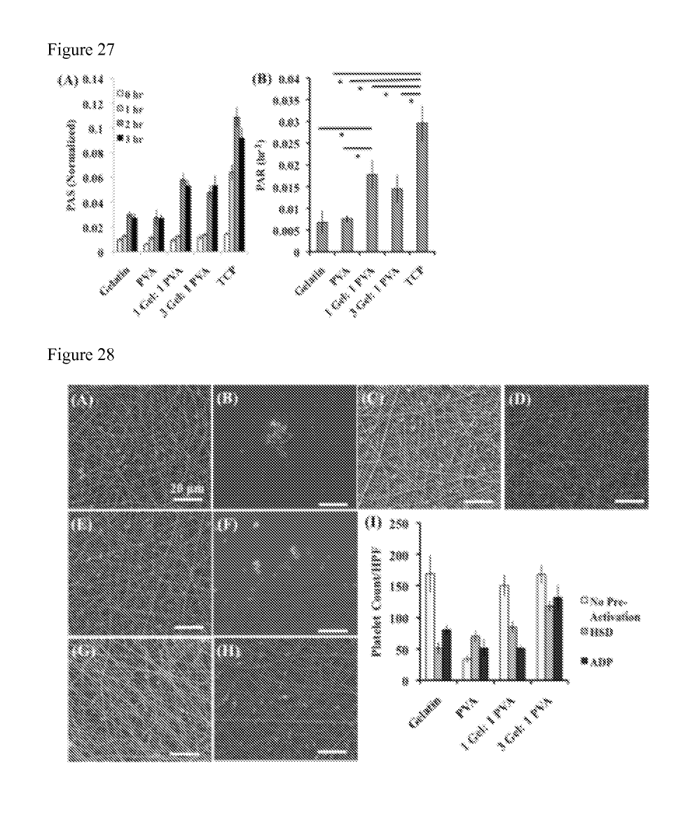

The PAS of the platelets on the scaffolds (gelatin, PVA, 1 Gel:1 PVA coaxial, and 3 Gel:1 PVA coaxial), as well as the tissue culture plate (TCP) are calculated and presented in FIG. 27A. The platelet activation rate (PAR) is calculated from the PAS values for each scaffold and the TCP over the 3 h of incubation, and this value was used for comparison between substrates. When the platelets were incubated on the scaffold or TCP surfaces, the platelet activation rate was highest for the TCP when compared with the gelatin scaffolds (p=0.004), PVA scaffolds (p=0.002), 1 Gel:1 PVA coaxial scaffolds (p=0.06), and 3 Gel:1 PVA coaxial scaffolds (p=0.03) (FIG. 27B). The coaxial scaffolds (1 Gel:1 PVA and 3 Gel:1 PVA) had similar PAR (p=0.52). The 1 Gel:1 PVA coaxial scaffold had higher PAR when compared to gelatin (p=0.045) and PVA (p=0.03); however, results were less significant when comparing the 3 Gel:1 PVA coaxial scaffolds with gelatin (p=0.12) or PVA (p=0.08).

The coaxial electrospun scaffolds are fabricated with PVA in the core and gelatin surrounding the PVA core. Therefore, gelatin, a product of collagen hydrolysis, comes into direct contact with the external environment, in this case, the platelets. Collagen, a triple helix protein, strongly promotes the adhesion and activation of human platelets (Cosemans, J. M. E. M. et al., Atherosclerosis 2005, 181, 19-27). For example, human platelets and collagen fibrils in suspension showed a pseudo-first-order kinetics adhesion profile reaching 60% adhesion at 60 min of incubation (Dolowy, K. et al., Collagen Relat. Res. 1984, 4, 111-118). Platelets can bind directly to collagen via integrin α2β1 and glycoprotein VI as well as indirectly to collagen through von Willebrand factor. Platelet interaction and binding with collagen is dependent on the platelet receptor recognizing the protein sequence. Taking this into consideration, gelatin, a single stranded polypeptide derived from collagen, has the protein sequences needed for platelet binding; however, the single stranded structure is not recognized by these same platelet receptors as it is for collagen. Despite this, there are additional protein sequences (RGD) that are exposed in gelatin's single stranded structure that do not contribute when in the triple helix structure of collagen that may bind to platelet sites such as α3β1, α5β1, αVβ3, and potentially αIIbβ3.57 Additionally, a study by Milleret et. al showed that fibers less than 1 μm in diameter had low platelet adhesion and coagulation (Milleret, V. et al., Acta Biomater. 2012, 8, 4349-4356). Therefore, the different mechanism of platelet interaction present in the gelatin compared to collagen, as well as the nanosized fibers may account for the low PAR observed in this modified prothrombinase assay.

Platelets are able to react to the mechanical properties of the underlying substrate. Thus, platelet adhesion, spreading, and subsequent activation is greater on the stiffer substrates than the softer materials (Qiu, Y. et al., Mechanosensing: Adhesion and Spreading on Immobilized Fibrinogen Depends on Substrate Stiffness, 54th ASH Annual Meeting and Exposition, Atlanta, Ga., Dec. 8-11, 2012). Previous studies have determined the mechanical properties of the gelatin scaffolds, PVA scaffolds, and the coaxial scaffolds (1 Gel:1 PVA and 3 Gel:1 PVA) (Merkle et al., Biopolymers 2014, 101, 336-346; Merkle, V.; et al., Polymers 2013, 54, 6003-6007). The coaxial scaffolds have the highest Young's modulus (3 Gel:1 PVA, 221±28.4 MPa and 1 Gel:1 PVA, 168.6±36.5 MPa) (Merkle et al., Biopolymers 2014, 101, 336-346; Merkle, V.; et al., Polymers 2013, 54, 6003-6007) and subsequently the highest PAR. In comparison to the coaxial scaffolds, gelatin has the lowest Young's modulus (21.52±4.15 MPa) (Merkle et al., Biopolymers 2014, 101, 336-346; Merkle, V.; et al., Polymers 2013, 54, 6003-6007) and the lowest PAR. The gelatin and coaxial scaffolds (1 Gel:1 PVA coaxial and 3 Gel:1 PVA coaxial) all have gelatin external that comes into direct contact with the platelets. Therefore, when comparing these three scaffolds (gelatin, 1 Gel:1 PVA coaxial and 3 Gel:1 PVA coaxial), the mechanical stiffness of the fibers predominates in platelet activation with the stiffer substrate having the highest activation rather than the biochemical signals of gelatin.

Gelatin has the lowest Young's modulus (21.52±4.15 MPa) and a similar PAR to PVA (p=0.30). PVA has a Young's modulus of 100.5±23.5 MPa; however, the PAR was low compared to the coaxial scaffolds and TCP. Although PVA has a high surface roughness and moderate stiffness compared to the gelatin and coaxial scaffolds, the PAR was low. In previous studies, it was shown that NIH 3T3 fibroblasts seeded on the PVA scaffolds had a round morphology with minimal attachment sites compared to the gelatin or coaxial scaffolds (1 Gel:1 PVA coaxial and 3 Gel:1 PVA coaxial scaffolds) that have gelatin in the shell of each nanofiber (Merkle, V. M. et al., Biopolymers 2014, 101, 336-346). This study showed that PVA fibers do not promote the fibroblast growth and proliferation that was seen on the gelatin and coaxial scaffolds.

It was hypothesized that this was due to the lack of cellular attachment sites that the gelatin and gelatin coated fibers (1 Gel:1 PVA coaxial and 3 Gel:1 PVA coaxial) have in abundance (Merkle, V. M. et al., Biopolymers 2014, 101, 336-346). A similar response was seen with the platelets having low deposition and activation on the PVA scaffolds in comparison to the gelatin and coaxial scaffolds. Overall, the PVA scaffolds do not promote cell growth and migration (Merkle, V. M. et al., Biopolymers 2014, 101, 336-346), as well as minimize platelet activation compared to gelatin scaffolds or the coaxial scaffolds. The modified prothrombinase assay measuring platelet activation shows that the mechanical stiffness of the underlying substrate is a dominating factor in comparison to surface roughness and biochemical signals.

Platelet Deposition of Chemically or Mechanically Activated Platelets.

Next, platelet deposition on the nanofiber surface that was preactivated through different mechanisms (mechanical or chemical activation) was determined. Deposition of platelets that were mechanically activated prior to incubating on the scaffolds are shown in FIG. 28A-D. The platelets were mechanically activated using a hemodynamic shear device (HSD) that exposes the platelets to dynamic shear stresses similar to the mechanical forces experienced in vivo (Nobili, M. et al., ASAIO J. 2008, 54, 64-72). Additionally, the HSD provides uniform and repeatable stress to the platelet samples (Sheriff, J. et al., Ann. Biomed. Eng. 2010, 38, 1442-1450; Nobili, M. et al., Eur. J. Vasc. Endovasc. Surg. 2006, 31, 627-636). In this study, the 3 Gel:1 PVA coaxial scaffold had the highest mechanically activated platelet deposition when compared to the gelatin scaffolds, PVA scaffolds, and 1 Gel:1 PVA coaxial scaffold (p<0.03) (FIG. 28I). This high platelet deposition correlated with the stiffness of the underlying substrate as discussed previously with the 3 Gel:1 PVA coaxial scaffold having the highest stiffness (Merkle, V. M. et al., Biopolymers 2014, 101, 336-346) and subsequent highest platelet deposition.

Then, platelets were chemically activated using adenosine diphosphate (ADP), a known platelet activator (Angiolillo, D. J. et al., Circ. J. 2010, 74, 597-607; Jennings, L. K. et al., Thromb. Haemostasis 2009, 102, 248-257). ADP activates platelets through a GTP-binding proteins or G proteins. This ADP-platelet interaction causes a platelet shape change and a decline in cAMP (cyclic adenosine monophosphate) formation, leading to platelet activation. Therefore, ADP is an important chemical factor at locations of vascular injury for the propagation of platelet activation (Woulfe, D. et al., J. Clin. Invest. 2001, 107). In this study, the deposition of chemically activated platelets deposited per high-powered field (HPF) on each scaffold was calculated (FIG. 28E-H). Similar to the mechanically activated platelets, the 3 Gel:1 PVA coaxial scaffolds had the highest chemically activated platelet deposition when compared with the gelatin scaffolds, PVA scaffolds, and 1 Gel:1 PVA coaxial scaffolds (p<0.03) (FIG. 28I). This high platelet deposition correlated with the high stiffness of the 3 Gel:1 PVA coaxial scaffolds (Merkle et al., Biopolymers 2014, 101, 336-346).

In this study, the preactivated platelets (both chemical and mechanical preactivation) had lower deposition on the scaffolds (gelatin, 1 Gel:1 PVA coaxial, and 3 Gel:1 PVA coaxial) than platelets that had no preactivation (FIG. 28I). Platelets preactivated either mechanically or chemically undergo numerous changes, including physical shape change, exposure/activation of surface receptors, as well as secretion of chemical agonists (Gregg, K. L. A Mathematical Model of Blood Coagulation and Platelet Deposition Under Flow. Ph.D. Dissertation, The University of Utah, Salt Lake City, Utah, 2010). Exposure and activation surface receptors plays a role in platelet aggregation (Gregg, K. L. A Mathematical Model of Blood Coagulation and Platelet Deposition Under Flow. Ph.D. Dissertation, The University of Utah, Salt Lake City, Utah, 2010). These preactivated platelets via chemical or mechanical means had less deposition on the scaffold surface (gelatin, 1 Gel:1 PVA coaxial, and 3 Gel:1 PVA coaxial) than the platelets that were not preactivated prior to incubation. It was contemplated that preactivation through chemical or mechanical means is causing platelet aggregation of the platelets in solution over the incubation period leading to less platelet deposition on the fiber surface for a given/fixed concentration of platelets.

Platelet Deposition on Electrospun Scaffolds Preseeded with SMC or HUVEC.

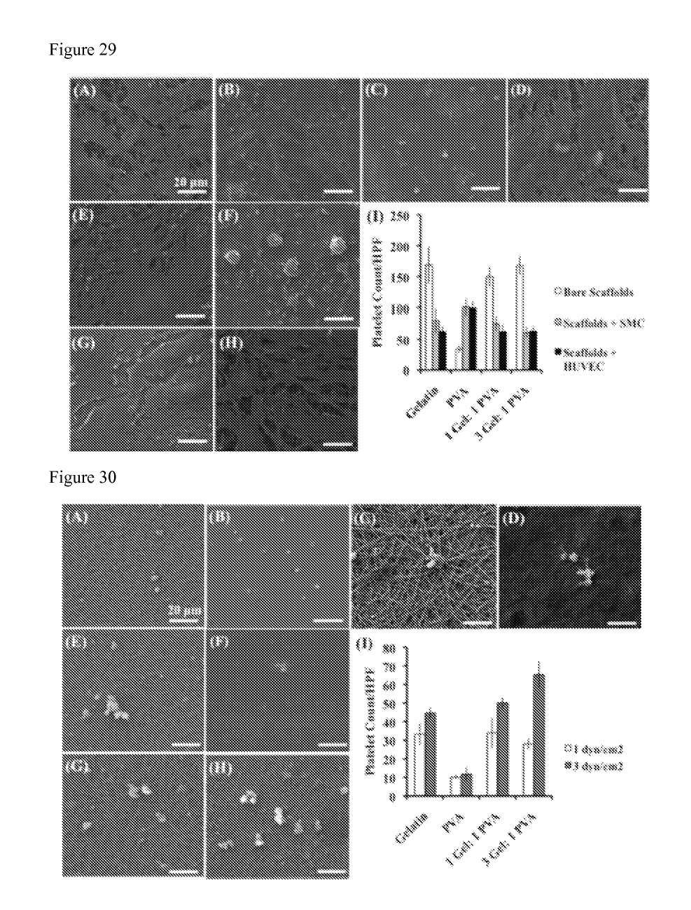

Once the scaffold is placed in the body, vascular relevant cells (such as endothelial cells and smooth muscle cells) will begin migrating and proliferating on the fibers. Therefore, platelet deposition on the nanofiber surfaces when they are covered with a monolayer of smooth muscle cells or endothelial cells was assayed. First, electrospun scaffolds were preseeded with smooth muscle cells (SMC) for 72 h to ensure a confluent monolayer of cells on the scaffold surface. Once the confluent monolayer of SMC was attained, platelets were incubated for 4 h on the scaffold surfaces prior to fixation and determination of platelet deposition (FIG. 29A-D). The PVA had similar platelet deposition as the gelatin scaffold (p=0.38) and 1 Gel:1 PVA coaxial scaffold (p=0.16), as well as significantly more platelets than the 3 Gel:1 PVA coaxial scaffold (p=0.02). Gelatin scaffolds had similar platelet deposition to the 1 Gel:1 PVA coaxial scaffold (p=0.22) and 3 Gel:1 PVA coaxial scaffold (p=0.39). Overall, there was less platelet deposition on the gelatin scaffolds (80±19 platelets) and the coaxial scaffolds (1 Gel:1 PVA coaxial, 75±12 platelets; 3 Gel:1 PVA coaxial, 61±9 platelets) preseeded with the SMC than on the scaffolds alone (without any cell preseeding). This was not true for the PVA scaffolds, which had higher platelet deposition with the preseeding of SMC than on the scaffold fibers alone (101±14 platelets with preseeding vs 34±6 platelets on fibers alone). The PVA nanofibers preseeded with the smooth muscle cells, which possessed a round morphology with only a few attachment sites to the underlying nanofibers, had the highest platelet deposition. In contrast, the smooth muscle cells on the gelatin and coaxial scaffolds possessed a flattened morphology with numerous attachment sites, decreasing the platelet deposition in comparison to scaffolds with no cell preseeding.

Electrospun scaffolds were preseeded with HUVEC for 72 h to allow the cells to proliferate and form a confluent monolayer. After the 72 h of incubation, the cells were incubated with platelets for 4 h and subsequently prepared for SEM imaging and counting (FIG. 6E-H). A similar trend was observed for the HUVEC preseeding as was seen with the SMC preseeding (FIG. 29I). The PVA had the highest platelet deposition (99±11 platelets), whereas the gelatin (62±9 platelets), 1 Gel:1 PVA coaxial (62±13 platelets), and 3 Gel:1 PVA coaxial (63±8 platelets) scaffolds had similar and lower depositions than platelet deposition on the scaffold fibers alone. Studies have shown the antithrombogenic properties of the native endothelium such as the release of nitric oxide (NO) and prostacyclin to decrease platelet activation and adhesion (Landmesser, U. et al, Circulation 2004, 109, II-27-II-33; Loscalzo, J. Circ. Res. 2001, 88, 756-762). Therefore, the scaffolds (gelatin, 1 Gel:1 PVA coaxial, and 3 Gel:1 PVA coaxial) that had a confluent monolayer of endothelial cells similar to the native vasculature displayed a decline in platelet deposition compared to the scaffolds with no endothelial preseeding. Scaffolds that were preseeded with smooth muscle cells also showed a decline in platelet deposition. After vascular injury, various mitogenic and chemotactic factors are released from the wound area, as well as from aggregated platelets that are on the damaged intimal surface. These factors initiate the neointimal response (Louis, S. F.; Zahradka, P. Exp. Clin. Cardiol. 2010, 15, e75-e85).

It was contemplated that in the absence of vascular injury, cell preseeding would reduce platelet deposition for gelatin and gelatin-coated fibers, as was observed for both endothelial cells and smooth muscle cells respectively in this experiment. In contrast, platelet deposition increased on the PVA scaffolds that were preseeded with endothelial cells or smooth muscle cells. Endothelial cells, smooth muscle cells, and/or fibroblasts produce components involved in the hemostatic response in vivo (Zwaginga, J. J. et al., Arteriosclerosis 1990, 10, 437-448). Previous studies have shown low cell attachment of 3T3 fibroblasts on the PVA scaffolds (Merkle et al., Biopolymers 2014, 101, 336-346). With low cell attachment at the time of platelet deposition, it was contemplated that the hemostatic balance was no longer maintained, thereby making these surfaces more thrombogenic than the PVA fibers alone. Overall, the formation of a confluent monolayer of SMC or HUVEC decreased platelet deposition than on the scaffolds alone, indicating that the nanofibrous scaffold has proadhesive and antithrombogenic properties for cells and platelets, respectively.

Platelet Deposition on Electrospun Scaffolds Under Flow.

Areas of low wall shear stress in the coronary arteries tend to have an increase in plaque accumulation, as well as an increase in necrotic core. The low wall shear stress results in a decrease in alignment of the endothelial cells to the flow axis, as well as an increase in low-density lipoproteins (LDL), smooth muscle cell proliferation, and macrophage migration (Samady, H. et al., Disease Circulation 2011, 124, 779-788). Therefore, understanding platelet deposition on the electrospun surfaces under flow conditions is useful for vascular applications. For this experiment, the scaffolds (gelatin, PVA, 1 Gel:1 PVA coaxial, and 3 Gel:1 PVA coaxial) were electrospun onto stainless steel chips, cross-linked with glutaraldehyde vapor, and placed into a parallel plate flow chamber (Bacabac, R. G. et al., J. Biomech. 2005, 38, 159-167). A pulsatile pump was used to circulate platelet-rich plasma (PRP) through the flow loop at either 1 dyn/cm2 (FIG. 30A-D) or 3 dyn/cm2 (FIG. 30E-H). After the PRP circulates through the flow loop, the scaffolds and stainless steel chips are removed, fixed, and prepared for SEM imaging (FIG. 30). Platelet counts per high-powered field (3000×) are calculated as previously described (FIG. 30I).

In a study conducted by Badimon et al., a de-endothelialized vessel wall showed an increase in platelet deposition with an increase in exposure time and shear (Badimon, L. et al., Arteriosclerosis 1986, 6, 312-320). Taking only flow rate into consideration, the increase in shear activates platelets mechanically, leading to an increase in platelet deposition (Colman, R. W. et al., Hemostasis and Thrombosis: Basic Principles and Clinical Practice, 5 ed.; Lippincott Williams & Wilkins: Philadelphia, 2006). Platelet deposition increased when the shear was increased from 1 to 3 dyn/cm² for the 3 Gel:1 PVA coaxial scaffolds (p<0.001) with less significance seen in the 1 Gel:1 PVA coaxial scaffolds (p<0.07) and gelatin scaffolds (p<0.09). It is contemplated that the mechanical shear stress not only induced platelet activation but also mobilized them toward the scaffolds, such that an increase in platelet deposition was observed with an increase in shear stress. Overall, platelet deposition under shear conditions was much less than static conditions. For instance, PVA had the least platelet deposition of all the scaffolds for the static conditions (FIG. 29I, bare scaffold), as well as the shear conditions (FIG. 30I, 1 or 3 dyn/cm2). This low platelet deposition for the static and shear conditions was attributed to the hydrophilic nature of PVA and poor cellular attachment and proliferative nature of the PVA scaffolds, Ikada et al. showed that there was less platelet deposition and fibrin formation using PRP than on nongrafted and acrylic acid-grafted polyethylene (Ikada, Y. et al., J. Biomed. Mater. Res. 1981, 15, 697-718). Overall, the platelet deposition was significantly decreased under shear than in static conditions, indicating that the scaffolds perform better under dynamic flow than in a static environment.

In this example, coaxial nanofibers composed of gelatin in the shell and poly(vinyl alcohol) (PVA) in the core of each fiber were prepared and the hemocompatibility was evaluated by determining platelet deposition and activation under varying conditions. It was contemplated that the coaxial nanofibers with gelatin in the shell and PVA in the core are an optimal construct for vascular applications, displaying minimal platelet deposition and activation. First, PVA nanofibers had the highest surface roughness (Ra) in comparison to gelatin and the coaxial scaffolds (1 Gel:1 PVA coaxial fibers and 3 Gel:1 PVA coaxial fibers). Despite the highest surface roughness, the PVA scaffolds had the lowest platelet deposition under static conditions out of all of the scaffolds (gelatin, PVA, 1 Gel:1 PVA coaxial, and 3 Gel:1 PVA coaxial). Therefore, the biochemical signals of gelatin dominated surface roughness for platelet deposition on these electrospun fibers. Next, the modified-prothrombinase assay was used to determine the rate of thrombin formation of platelets on the scaffolds. Overall, the coaxial scaffolds had the highest platelet activation rate of the electrospun fibers, which was followed by gelatin and PVA scaffolds. The increase in platelet activation rate correlated with the increase in stiffness of the underlying fibers. Therefore, platelet activation of fibers with the same surface chemistry (e.g., gelatin external) is dominated by mechanical stiffness of the underlying substrate. The experiments show that the following factors influence platelet deposition and activation on the fibers in order significance: mechanical stiffness followed by biochemical signals, and lastly surface roughness. Additionally, when the scaffolds were preseeded with either SMC or HUVEC, the platelet deposition decreased significantly on the gelatin and coaxial scaffolds. In contrast, preseeding with the SMC or HUVEC increased platelet deposition on the PVA scaffolds. Overall, the 1 Gel:1 PVA coaxial scaffolds, promoting cellular viability and growth, as well as minimal platelet deposition and activation, possess appealing hemocompatibility for use in vascular applications.

All publications and patents mentioned in the above specification are herein incorporated by reference as if expressly set forth herein. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the invention.

We claim:

1. A composition, comprising:
a multi-layer nanofiber comprising a polymeric core and a biocompatible shell.

2. The composition of claim 1, wherein said polymeric core is comprised of a material selected from glycolic acid polymers, lactic acid polymers, polyurethanes, polyesters such as poly(ethylene terephthalate), nylon, polyacrylonitriles, polyphosphazines, polycaprolactone, poly[bis(p-carboxphenoxy)propane anhydride], polyethylene, polyvinyl chloride, ethylene vinyl acetate, homopolymers and copolymers of delta-valerolactone, and p-dioxanone, and polyvinyl alcohol.

3. The composition of claim 1, wherein said biocompatible shell is comprised of a biocompatible material selected from gelatin, collagen, fibrin, fibrinogen, albumin, laminin, zein, lipids, phospholipids, and glycoproteins.

4. The composition of claim 3, wherein said biocompatible material comprises an agent that alters the surface texture or functionality of said nano-fiber.

5. The composition of claim 4, wherein said agent is selected from ligands, nanoparticles, iron, labels, contrast agents, cells, encapsulated particles, and viruses.

6. The composition of claim 4, wherein said agent is incorporated into said biocompatible material or on the surface of said biocompatible material.

7. The composition of claim 1, wherein said nanfiber is a core-shell nanofiber comprising a polyvinyl alcohol core and a gelatin shell.

8. The composition of claim 7, wherein said PVA and said gelatin are present at a ratio of approximately 1:1 in said nanofiber.

9. The composition of claim 7, wherein said PVA and said gelatin are present at a ratio of approximately 1:3 in said nanofiber.

10. The composition of claim 1, wherein said nanofiber is made by electrospinning.

11. The composition of claim 1, wherein said nanofibers exhibit an increased Young's modulus, a higher tensile strength, and reduced plastic deformation relative to PVA nanofibers.

12. A system, comprising:
a) a core-shell nanofiber of claim 1; and
b) a plurality of cells, tissues or an organ in operable communication with a surface of said nanofiber.

13. The system of claim 12, wherein said cells are selected from stem cells, islet cells, fibroblasts, hormone secreting cells, neurons, epithelial cells, and blood cells.

14. The system of claim 12, wherein said tissues are selected from connective tissue, muscle tissue, nervous tissue, and epithelial tissue.

15. The system of claim 12, wherein said organ is selected from heart, stomach, liver, gallbladder, pancreas, intestines, colon, rectum, anus, hypothalamus, pituitary gland, pineal body or pineal gland, thyroid, parathyroids, adrenals, kidneys, tonsils, adenoids, thymus, spleen, skin, hair, nails, brain, spinal cord, nerves, ovaries, fallopian tubes, uterus, vagina, mammary glands, testes, vas deferens, seminal vesicles, prostate, penis, pharynx, larynx, trachea, bronchi, lungs, diaphragm, bones, cartilage, ligaments and tendons.

16. A method of culturing cells, tissues, or organs, comprising:
a) contacting a cell, tissue, or organ with a surface of a nanofiber of claim 1; and
b) culturing said cell, tissue, or organ.
